(19) United States
(12) Patent Application Publication
(10) Pub. No.: US 2024/0115875 A1
(43) Pub. Date: Apr. 11, 2024

(54) Systems, Methods, and Substances for Controlled Endoluminal Energy Application

(71) Applicant: Dream Big Biotechnologies B.V., EC Nijmegen (NL)

(72) Inventor: Arjen BOGAARDS, Forchheim (DE)

(73) Assignee: Dream Big Biotechnologies B.V., EC Nijmegen (NL)

(21) Appl. No.: 18/486,376

(22) Filed: Oct. 13, 2023

Related U.S. Application Data
(63) Continuation-in-part of application No. PCT/EP2022/059771, filed on Apr. 12, 2022.
(60) Provisional application No. 63/272,935, filed on Oct. 28, 2021.
(60) Provisional application No. 63/175,743, filed on Apr. 16, 2021.

(51) Int. Cl. A61N 5/06

(52) U.S. Cl. CPC A61N 5/062; A61N 2005/0602; A61N 2005/0627

(57) ABSTRACT

Systems and methods for isolating a biological vessel from a surrounding target tissue include administering an energy-activatable substance to at least a portion of the target tissue and activating the energy-activatable substance via intraluminal irradiation of the portion of the target tissue with the energy-activatable substance, preferably while keeping the vessel wall substantially intact.

### CROSS-REFERENCE TO RELATED APPLICATIONS

This application is a bypass continuation-in-part application of PCT Application No. PCT/EP2022/059771, filed Apr. 12, 2022, which claims priority to U.S. Provisional Pat. App. No. 63/175,743, filed Apr. 16, 2021, both of which are herein incorporated by reference in their entireties.

### TECHNICAL FIELD

The following relates to systems for freeing a vessel from surrounding tissue and more particularly to systems and methods that utilize an energy-activatable substance and an energy-emitting element to free a vessel from surrounding tissue, preferably while leaving the vessel wall substantially intact.

### BACKGROUND

Often, the most effective form of treating a cancerous tumor within a subject is to surgically remove the tumor. For some advanced types of cancer, the tumorous tissues have invaded around a blood vessel which renders surgical resection unfeasible. In some cases, this greatly reduces a patient's chances of survival.

### SUMMARY

Aspects of the present disclosure address the above-referenced problems and/or others.

In one aspect, a method for isolating a biological vessel from a surrounding target tissue, preferably while leaving the vessel wall substantially intact, includes administering an energy-activatable substance to at least a portion of the target tissue and activating the energy-activatable substance via intraluminal irradiation of the portion of the target tissue with the energy-activatable substance.

In another aspect, a system includes an energy-emitting element and an endoluminal wall protection device. The energy-emitting element is configured to be placed within a vessel of a subject and is configured to emit radiation through the vessel to a target tissue external to the vessel. The endoluminal wall protection device configured to be placed within the vessel and configured to provide structural support to the irradiated portion of the vessel.

In another aspect, a method of treating a patient suffering from cancer includes administering an energy-activatable substance to at least a portion of the target tissue of an organ, wherein the at least a portion of the target tissue is in contact with a portion of an external surface of a vessel of the organ; activating the energy-activatable substance via intraluminal irradiation of the portion of the target tissue containing the energy-activatable substance to effect isolation of at least a part of the at least a portion of the target tissue from the vessel surface; and causing any one or more or any combination of removal or necrosis of at least some of the isolated part of the at least a portion of the target tissue to treat cancerous tissue.

### BRIEF DESCRIPTION OF THE DRAWINGS

Aspects of the present disclosure may take form in various components and arrangements of components, and in various steps and arrangements of steps. The drawings are only for illustration purpose of preferred embodiments of the present disclosure and are not to be considered as limiting.

Features of embodiments of the present disclosure will be more readily understood from the following detailed description take in conjunction with the accompanying drawings in which:

FIG. 1 is a perspective view of a human pancreas and adjacent arteries with a stent-like implant according to an embodiment of the disclosure deployed in the superior mesenteric artery (SMA), or arteria mesenterica superior;

FIGS. 2A-2D show a system and some of its components for applying energy to a target tissue according to some embodiments;

FIGS. 3A-3c show some components of the device and the corresponding energy distribution profile curves according to three different embodiments;

FIGS. 4A and 4B are perspective views of a system according to another embodiment;

FIG. 5 is a perspective view of a body-part and a stent-like implant according to another embodiment being deployed there; and

FIGS. 6A-6C show different views of a system for the transfer of energy according to another embodiment.

FIG. 7A shows a pancreatic model used for dose simulation.

FIG. 7B shows an exemplary dose attenuation curve with an estimated necrosis radius outside the vessel wall in normal tissue (dark grey) and tumor tissue (light grey), with estimated necrotic margins ranging from 4-7 mm.

FIG. 8A shows a sketch of the pancreas. On the left is the adjacent duodenum curving around the head of the pancreas. On the right one can see the tail of the pancreas. The splenic artery running horizontally atop of the pancreas. The square indicates the endovascular PDT treatment location where the tissue sample was taken with similar orientation, plane and orientation as the surgically removed pancreas in FIG. 8B, the CT scans in FIG. 8C and the histopathological slide with H&E stain shown in FIG. 8D. FIG. 8B shows a picture of the resected porcine pancreas. On the left is the adjacent duodenum curving around the head of the pancreas. On the right one can see the tail of the pancreas. Indicated with the dotted line is the location of the splenic artery running horizontally atop of the pancreas. The square indicates the endovascular PDT treatment location, where a tissue sample was taken with similar plane and orientation as the sketch FIG. 8a, the CT scans in FIG. 8c and the histopathological slide with H&E stain shown in FIG. 8D.

FIGS. 9A-F shows contrast-enhanced CT scan pre- and post-treatment in the early portal venous phase. The crosshair indicates the position of treated target vessel being the splenic artery shown in coronal, sagittal and transverse view. In the post-treatment scans hypodense areas can be identified circumferentially around the treated vessel and are indicated with white arrows. These cannot be found in pre-treatment scans and are interpreted as possible PDT-induced necrosis within the pancreas. The square in FIG. 9B indicates the location where the tissue sample was taken with similar plane and orientation as the pancreas in FIGS. 8A-B, and the histopathological slide with H&E stain shown in FIGS. 10A-E.

FIG. 10A shows a post-treatment (48h) H&E stained histopathology sample. FIG. 10A shows an overview with the splenic artery running horizontally atop of the pancreas taken within the square shown in FIGS. 8A-D and 9A-F having similar size, plane and orientation. The pancreas (i) is demonstrating focal areas of necrosis, fat necrosis and apoptosis, indicated with several * Asterix signs, radially extending outwards up to circa 6 mm from the treated vessel. The adjacent lymph node (ii) and nerve tissue (iii) similarly demonstrate multifocal liquifying and hemorrhagic necrosis. Higher magnification detail is shown in FIG. 10B-E. When examining the splenic arterial wall (iv) in FIG. 10E, endothelial loss and necrotic cellular tissues were observed, but the extracellular matrix containing collagen and elastin was preserved in some cases, essentially creating an acellular or decellularized vascular scaffold (FIG. 10E).

The scale bar in FIG. 10A is 1 mm, and in FIGS. 10B-E is 100 μm.

FIG. 11A shows pre-treatment contrast enhanced CT demonstrating a sagittal view of a porcine pancreas for before an administered S-size dose (0.4 mg/kg) of verteporfin.

FIG. 11B shows post-treatment contrast enhanced CT demonstrating a sagittal view of the porcine pancreas of FIG. 11a after an S-size administered dose (0.4 mg/kg) of verteporfin.

FIG. 11C shows pre-treatment contrast enhanced CT demonstrating a sagittal view of a porcine pancreas before an administered M-size dose (0.8 mg/kg) of verteporfin.

FIG. 11D shows post-treatment contrast enhanced CT demonstrating a sagittal view of the porcine pancreas of FIG. 11C after an M-size administered dose (0.8 mg/kg) of verteporfin.

FIG. 11E shows pre-treatment contrast enhanced CT demonstrating a sagittal view of a porcine pancreas before an administered L-size dose (1.6 mg/kg) of verteporfin.

FIG. 11F shows post-treatment contrast enhanced CT demonstrating a sagittal view of the porcine pancreas of FIG. 11E after an L-size administered dose (1.6 mg/kg) of verteporfin.

FIGS. 12A-C shows a post-treatment H&E and EVG stained histopathology sample post-PDT treatment (48h) after an L-size administered dose (1.6 mg/kg) of verteporfin.

### DETAILED DESCRIPTION

As used herein, the term “about” means plus or minus 10% of the numerical value of the number with which it is being used. Therefore, about 100 μm means in the range of 90 μm-110 μm.

As described in further detail below, a vessel may be referred to as being “substantially intact.” As used herein, a vessel is “substantially intact” when the vessel is able to provide at least part of its physiological function. For example, largely maintaining its mechanical strength after the application of energy, in particular maintaining its mechanical strength after the application of energy while at the same time cellular components may be damaged or compromised and/or extracellular components may be less susceptible to damage and may be less compromised. Another example is that the vessel is not compromised to such a degree that it leaks, or relevantly leaks, and/or is perforated and/or is ruptured. A further example is that the vessel remains substantially patent, preferably without substantial stricture formation and/or substantial formation of thrombi.

The present disclosure is generally related to methods, systems, and substances for delivering energy (e.g., radiation) via an energy source (e.g. a radiant energy source) introduced into a subject's lumen to tissue that is external to the lumen while ensuring that the integrity of the lumen is maintained and variations in the intensity of radiation applied to different portions of the tissue are minimized. As discussed further below, in some embodiments the radiated tissue is diseased tissue, e.g., cancerous tissue. In some embodiments, an endoluminal wall protection device is implanted in the internal wall of the lumen to maintain the structural integrity of the lumen. The endoluminal wall protection device may protect the vessel from inadvertent damage. In one embodiment, the endoluminal wall protection device is a stent-like device. The stent-like device may be configured to have a radiation transferring/conducting element, such that radiation can be transferred through portions of the stent-like device and directed to the external tissue. In some embodiments, the radiation-transferring/conducting element through which radiation passes is configured to have optimized properties to reduce both a shadowing effect that can be caused by opaque portions of the stent as well as to reduce radiation “hot spots,” which may be generated in the absence of the radiation-transferring/conducting element. In some embodiments, these optimized properties, in the case of radiant energy, may be realized for a given energy spectrum, by selecting device materials with an optimized balance between transmittance and scattering properties, relative to the surrounding biological structures. In some embodiments, once the irradiation of the external tissue is completed, the energy source can be retracted from the lumen while the stent-like device is left behind. In other embodiments, the energy source can be incorporated in the stent and can be coupled, decoupled, and removed as desired. In some embodiments, the energy may activate a substance sensitive to such energy to achieve the desired effect.

In other embodiments, the endoluminal wall protection device utilizes a temporary vascular compression method to temporarily compress the vasa vasorum and deprive the vessel wall from oxygen which may prevent the oxygen dependent damage to the vessel. For example, the endoluminal wall protection device may be a balloon catheter with a slightly oversized balloon diameter (2-20%, optionally 10-20%) vs. the vessel wall.

In another embodiment, the endoluminal wall protection device utilizes energy-induced collagen cross-linking in the vessel wall, creating an in-situ vascular scaffold. This cross-linking may maintain or preferably strengthen the mechanical strength of the vessel wall. This cross-linking of collagen may be induced by light applied to an energy (e.g., light) activatable photosensitizer.

In some embodiments, the disclosure deals with the problem that, for some advanced forms of cancer, tumorous tissues have invaded around blood vessels. This makes complete surgical resection with free margins unpractical. This is a large clinical problem especially in relation to pancreatic cancer. Extensive vessel involvement causes patients to be classified as unresectable—even if the disease is confined to the pancreas and has not metastasized.

Although there are several types of pancreatic cancer, the most common is pancreatic adenocarcinoma, which appears in about 90% of pancreatic cancer cases. For this reason, the term “pancreatic cancer” is sometimes used to refer only to that type of cancer. These adenocarcinomas start within the caput pancreatis, being the part of the pancreas responsible for producing digestive enzymes.

If pancreatic cancer develops, cancerous cells in the pancreas often proliferate aggressively and tend to form metastases outside of the pancreas rapidly, limiting available therapeutic options and the chances for survival.

However, in a subset of patients, the disease is locally confined within the pancreas, without such distant metastases. Here, in some cases, surgical removal of the afflicted pancreatic tissue may be attempted.

In this regard, challenges to the technical success rate of such surgery are likely to arise if cancerous tissues are located in the direct vicinity of a blood vessel, especially near the arteries. Three-dimensional (3D) medical imaging modalities such as computed tomography (CT) or magnetic resonance imaging (MRI) may be used to visualize if tumorous tissues are abutting large blood vessels. If abutment is identified, then a complete surgical resection with tumor free margins may be unlikely, due to the aforementioned high complexity of the anatomical structures. As a result, about one third of patients diagnosed with pancreatic cancer are deemed inoperable because of such large vessel involvement and surgical resection is not attempted.

It would be beneficial if there were some form of therapy that could clear blood vessels from such tumor involvement that could effectively create circumferential tumor free margins around these vessels, isolate the vessels from the affixed surrounding tumorous tissues, such that patients may be down-staged, become eligible for surgery, and ultimately have better chances for improved survival. Several downstaging approaches are being investigated, e.g. combinations of systemic chemotherapy and radiation therapy. Results are encouraging, however, at the cost of local and systemic toxicities. Not all patients are down-staged effectively, and the typical treatment duration is in the order of weeks to months before surgery. Thus, the development of a more effective local treatment modality with short treatment response duration is highly desirable. Various embodiments enable application of energy via the endovascular route to circumferentially damage (e.g., ablate) tumor tissue surrounding the vessel while leaving the structural integrity of the vessel intact. The approach may work optimally for ablation technologies with a non-heat based mechanism of action, such as brachytherapy, cryotherapy, electrochemotherapy and (irreversible) electroporation. In particular, in some embodiments, the present teachings may employ photosensitizing agents used in photodynamic therapy to cause selective damage to diseased tissue (e.g., cancerous tissue) via photochemical reactions initiated by irradiation of the photosensitizing agents, while leaving the extracellular matrix providing structural support to a vessel through which activating radiation is applied to the photosensitizing agents intact.

In some embodiments, a local ablation method with the following capabilities may be provided:
(1) treatment of the tumor tissues around the blood vessels, circumferentially, from inside the blood vessel;
(2) creation of a tumor free margin of circa 2-6 mm outside of the blood vessel without substantial damage to the vessel itself; and
(3) being readily visible with standard imaging techniques such as CT and MRI.

Deployment of this method would thereby potentially isolate the vessel from the affixed surrounding tissues and render this otherwise inoperable patient population eligible for surgery. In some illustrative use cases, this method could also be applied at later cancer stages to manage malignant obstructions, aiming to palliate, minimize and delay the complications caused by tumor invasion into bodily vessels and conduits, such as the bile duct.

This type of treatment could be relevant for a variety of diseases, including cancers of various forms including, but not limited to, pancreatic cancers, central tumors within the liver with proximity to vessels, gall bladder cancers, bile duct cancers, colorectal cancers, urothelial cancer, transurethral treatment of benign prostatic hyperplasia, renal denervation, applications in the urinary and gastrointestinal tract, urinary infections and pathogen inactivation within or in the proximity of biological conduits or ducts. Especially, to treat certain cancer patients with invasive extravascular tumor tissue in proximity or contact with a vessel. It may also find use in immunotherapeutic approaches, for example to optimize antigen presentation and priming of the immune system.

Photodynamic therapy (PDT) is a local ablation technique with a photochemical, non-thermal mechanism of action. PDT may require the administration of a light sensitive drug, known as a photosensitizer. Sometime after administration, this drug may be selectively taken up in cancerous tissues to a higher extent compared to the surrounding normal tissues. The photosensitizer itself may be non-toxic and may be activated by illumination with an activation light having a particular wavelength capable of triggering a photochemical reaction. In the presence of oxygen, the photochemical mechanism of action may generate cytotoxic oxygen radicals that may cause tumor cell death. These oxygen radicals may be short lived, and the treatment effect (e.g. hemorrhagic necrosis) may therefore be locally confined to the area where the activation light was applied. Observations of PDT in the proximity of large vessels do not appear to show impact on blood flow or cause thrombus formation. Moreover, PDT may leave the extracellular matrix (ECM) of biological tissues including large blood vessels intact. In this regard, the ECM provides the mechanical support structure of a vessel with a fibrous network of collagen, elastin, etc. As such, PDT is a modality with probability to cause substantial tissue damage in the (tumorous) tissues surrounding the vessel, yet while leaving the ECM of the vessel wall itself substantially intact. This is rather contrary and counterintuitive in comparison with commonly used ablation technologies (e.g. radiofrequency and microwave ablation) as these are known for their ability to cause damage to blood vessels. As such, in some embodiments, PDT may be used to ablate tumor tissues outside of large blood vessels via the endovascular route, while leaving the ECM of the vessel wall intact. Doing so, may isolate the vessel from its surrounding affixed tissues to facilitate surgical resection around such vessel. In other embodiments other activatable substances may be administered to a subject. These substances, like photosensitizers, may be activated to cause tumor cell death. These activatable substances, hereinafter referred to as “energy-activatable substances,” include, but are not limited to, photosensitizers, photocatalysts (e.g. TiO2) that may be activated by energy from photons, radiosensitizers (e.g. Nimorazole) which may be activated by energy from radiation, magnetic particles (e.g. Magnetite) that may be activated by magnetic energy (e.g. utilizing electromagnetic coils) and a variety of nanomaterials (e.g. Quantum Dots) which may be activated with radiant energy such as light. Photosensitizers include, but are not limited to verteporfin, talaporfin, padeliporfin, and temoporfin. Energy-activatable substances may further be administered in the form of a prodrug, for example, 5-aminolevulinic acid induced protoporphyrin IX.

In some embodiments, cell death may be apoptosis, autophagy, necrosis, e.g. hemorrhagic necrosis, or entosis.

In some embodiments, the energy-activatable substances may be rather passive and non-actively targeted, or, actively targeted. This may be achieved for example by conjugation of the energy-activatable substance to a targeting moiety that specifically binds to a moiety of a target cell (e.g., a cancerous cell, or part thereof within a target tissue) and may be a part of a drug delivery system, a multi-functional nanoparticle system, it can be based on a liposomal drug delivery system (e.g. poly(ethylene glycol)—polylactide micelles), nanomaterials, nanoparticles (e.g. quantum dots and gold nanoparticles), PEGylationand the like. The targeting moieties may include, but are not limited to, antibodies (e.g., Anti-EGFR, Anti-HER2 and Anti-VEGF), antibody fragments (e.g. Ranibizumab and Certolizumab pegol), peptides (e.g. iRGD, a 9-amino acid cyclic peptide), aptamers (e.g. pegaptanib), small molecules (e.g. organic compounds with a low molecular weight of less than about 900 Daltons) and the like.

Providing energy-activatable substances with targeting moieties may allow the energy-activatable substances to accumulate in a tumor at high concentrations as the energy-activatable substances may specifically bind to cells within the tumors which may provide for better targeting between healthy and diseased cells. The energy-activatable substances may be administered orally, intra-arterially, intravenously or injected directly into a target tissue. In some embodiments, the energy-activatable substance is administered so that it accumulates in the target tissue, for instance in the tumor tissue.

Other non-heat based ablation technologies of interest include, for example, brachytherapy, cryotherapy, electrochemotherapy and irreversible electroporation. These may for example be implemented via a percutaneous needle-based approach, or via the endovascular route.

Some of the embodiments provide a system for the transfer of energy emitted within the lumen of a bodily vessel or biological conduit to target tissues that are external to the bodily vessel or the biological conduit by directing the energy outwards toward the wall of the vessel or conduit, such that at least a portion of the energy passes through the wall for the application to the target tissues. In these embodiments, the system comprises a stent-like implant that is tubular in shape and comprises, at least in part, an energy conducting element with a predefined energy transfer function and has a docking component capable of docking (the “docking capability”) with at least one separate pairing device. One or more pairing devices may have the ability of being functionally engageable with or releasable from the stent-like implant or the energy conducting element.

In some embodiments, the methods and devices allow for endovascular energy application by irradiation through an endovascular tubular implant with energy conducting properties, for example, for the treatment of extravascular cancerous target tissues.

One embodiment of the disclosure relates to a device and a method to safely perform local ablation therapy (e.g., photon therapy) via the endovascular route. Some embodiments provide an endovascular tubular implant having stent graft like properties with docking ability for one or more separate pairing devices. Pairing devices that may be coupled and uncoupled to the tubular implant are (i) an implant delivery device, (ii) an energy emitter, or (iii) an imaging and sensory probe. Here, the tubular implant comprises an energy conducting element with a design that allows that the energy transfer function of the energy conducting element may be defined during manufacturing in a desired way in order that a controlled energy dose will be delivered to the target tissues. The stent design is used to keep the vessel structure open in order to maintain and support fluid flow and to address the risk of vasoconstriction and vasodilation. The graft aspect is used to supplant damaged or compromised tissues to address the risk of vessel rupture and bleeding, whereas the energy emitter is used to supply a sufficient energy dose. In some embodiments, such implant is preloaded onto a delivery device and the energy is emitted from within the implant delivery device, enhancing the workflow and avoiding multiple device exchanges. In another embodiment, an energy emitter and stent-like implant are loaded onto a single delivery device, allowing for the application of energy and deployment of the stent-like implant in sequence, yet while minimizing multiple device exchanges. In some embodiments, the system and devices may have a bending radius in the range of circa 1 mm-40 mm to allow for placement and use in tortuous vessel structures. In some embodiments, the system and devices may have variable and user-controlled diameters, such that a single device can accommodate a range of vessel diameters.

The methods and devices for treatment may result in tissue destruction (e.g. via hemorrhagic necrosis) of extravascular cancerous tissues while leaving the vessel media and vascular wall ECM unharmed and intact with sufficient mechanical strength to perform their clinical functions.

The embodiments of the disclosure may address several risks in endovascular ablation therapy such as possible stricture, vasoconstriction, vessel perforations, rupture, and bleeding. To mitigate these risks, embodiments of the disclosure may include the use of a “safety belt” in the form of tubular implant with stent like properties, which may have energy compatible/resistant properties and may also have the ability to transmit energy throughout the implant via an energy conducting element.

The implant may have stent-graft like properties, having the function to maintain the mechanical integrity of the vessel by supporting a potentially damaged vessel wall, as well as keeping the lumen open as to allow for natural flow tissues.

In some embodiments, the tubular implant further has energy conducting properties, operable to transmit a sufficiently high dose of energy through the stent and direct these toward the target tissues.

Furthermore, the tubular implant according to some embodiments of the disclosure has the ability to function as a docking station for one or more separate pairing devices, for example including, but not limited to, an implant placement/delivery device, an energy emitter, and optionally a sensory/imaging probe. Pairing devices may be coupled via a catheter or similar construct to an intra- or extra-corporeal energy source, sensory probe ready out, or fluid source.

Moreover, some embodiments may utilize a purpose-built design element capable of modifying and customizing i) the energy transfer function as well as ii) the spatial physical dimensions of the implant that is tailored to the anatomy of the target tissues.

Some embodiments provide a comprehensive endovascular energy-based therapy system, where the stent-like implant, the implant placement/delivery device, the energy emitter, or the energy source have a predefined energy emission profile allowing for accurate and controlled dose estimation arriving at one or more target regions at which the target tissues are located, and treatment planning analogous to radiotherapy.

The resulting treatment effect (e.g. tissue necrosis) around the intact vessel may be seen as a circumferential resection of extravascular cancers and may be visualized as a ˜2-6 mm wide ring or halo around the vessel's cross-section, using standard cross-sectional imaging techniques (e.g. MR and CT). The ˜2-6 mm wide ring around the vessel provides a separation between the vessel and the cancerous tissue. This separation may allow for future surgical resection of the tumor that otherwise would not be possible.

The resulting tissue necrosis may result in vessel isolation from the affixed surrounding tissues and a tumor free margin around the largely intact vessel—such that pancreatic cancer patients traditionally deemed inoperable are effectively down-staged, and may become surgical candidates, ultimately aiming to improve their chances for survival.

The stent-like-implant providing mechanical support may, for example, have a tubular structure, so that it may be placed juxtaposed against the vessel wall. Some embodiments of the disclosure make use of non-expandable materials, similar to a plastic biliary stent, or the use of collapsible and self-expanding materials, or expandable material via a radial force (e.g. a balloon). In some embodiments, the stent-like implant may have a metal support structure and a polymer covering inside, outside, or surrounding the support structure. It may have, for example, pig tail shaped ends, perfusion holes, anchoring flaps, or barbs to avoid device migration within the lumen. Some embodiments may include materials that are bioabsorbing, bioresorbing, biodegrading, or drug eluting. Some embodiments may have one or more fenestrations, possibly for connecting multiple stent-like implants via these fenestrations.

In some embodiments, the stent-like implant is further removable from the body sometime after implantation. For example, it may be a knitted stent that may be unraveled for removal, or may comprise a connected wire, thread, tube, or extension by which it may be removed from the body. In some embodiments, it may have any other features of the like that are associated with stents and stent-grafts.

The presence of energy conducting properties may cause that irradiation (i) originate from within the implant; (ii) be transmitted through at least a portion of the implant; and (iii) be transmitted substantially unrestrictedly toward the target area located outside the implant.

A predefined energy transfer function of the energy conducting element may allow for an accurate estimation of an energy dose arriving at the target tissues, allowing for improved prediction of the treatment effect, and treatment planning analogous to radiotherapy. In some embodiments, the word ‘predefined’ may mean having specific design specifications for, or having a priori knowledge of, the devices disclosed herein with respect to their energy transfer function and the relevant properties that govern them, including scattering, absorbing, reflecting, or refracting properties as well as other properties.

In some embodiments, the energy conducting element absorbs less than about 30%, less than about 20%, less than about 10%, or less than about 5% of the energy, e.g. light, emitted by the energy-emitting element. In some embodiments, the energy conducting element transmits more than about 70%, more than about 80%, more than about 90%, or more than about 95% of the energy, e.g. light, emitted by the energy-emitting element.

Furthermore, the tubular implant may have the ability to function as a docking station to couple and decouple at least one separate pairing device, which has the ability of being engageable, for example, functionally engageable, with or releasable from the energy conducting element. Associated coupling and decoupling mechanisms may include: a locking and unlocking mechanism, a compression and decompression mechanism, or an O-ring compression. Such mechanisms may be based on one or a combination of: springs, pre-shaped forms, notches, a sliding mechanism, high friction surfaces, indentations, raise, bump, channels, tracts, rails, rings, loops, sutures, stitches, knots, magnetic parts, controlled breaking, combinations of loops and retractable wires, barbs, mechanical stoppers, and the like. The pairing device, for example, an implant delivery device or an energy-emitting device, may enable some embodiments to leave the stent-like implant in situ. The exemplary implant delivery device may also allow for treatment and re-treatment with a separate energy emitter that may be paired with the implant during treatment and removed thereafter. Similarly, it may also allow for the pairing device to be a sensory or imaging probe.

The separation of implant and pairing device may bring the intended advantage that the implant may remain in place for a prolonged period of time to support and protect the vessels, whereas the pairing devices, for example an energy emitter, may allow for initial treatment and subsequent retreatment with devices that may be removed from the body when they are not used. In some embodiments, the stent like implant provides a structural support for the vessel.

Within the scope of the disclosed system is the incorporation of advanced image guidance, tracking, navigation, and robotic methods. For example, but not limited to, MRI, CT, positron emission tomography (PET), single photon emission computed tomography (SPECT), angiography, ultrasound, optical imaging, endoscopy, bronchoscopy, colonoscopy, or combinations thereof, navigation systems (e.g. electromagnetic-based navigation, radio frequency identification (RFID)-based tracking, or optically based navigation methodologies) as well as robotic guidance systems, including the utilization of co-registration and fusion methodologies. These may for example be used for the optimal positioning of any of the devices claimed herein.

The implant and pairing devices may have access into the body via natural orifices or via a percutaneous access route and may include use of guidewires, guiding catheters, pushers, sheaths, balloons, and related endovascular devices. The implant and pairing devices disclosed herein may be useful for the application of energy, used for treatment of target tissues surrounding a vessel or conduit, while at the same time, acting as a stent for opening or strengthening such vessels or conduits, or as graft to support or replace compromised or damaged vascular wall, conduit or some other biological tissues having a lumen, including but not limited to portions of the vascular system, biliary, urinary, esophageal, digestive, ocular, tracheal, bronchial, reproductive, or neural systems. Intended uses may include uses that require the application of energy in a vessel or biological conduit, including but not limited to:
a) Therapeutic methodologies aiming for local tissue destruction, such as photochemical, thermal, and ionizing local ablation technologies—including:
b) Phototherapy, photon therapy, photodynamic therapy, brachytherapy, or photochemical internalization;
c) Ultrasound ablation, high intensity focused (HIFU), or unfocussed (HIU) ultrasound ablation;
d) Radiofrequency ablation, Microwave ablation, or cryoablation;
e) Electrosurgery, electrocautery, electrochemotherapy, reversible, or irreversible electroporation;
f) Modulation of the immune system; or
g) Sensing or detecting of, for example, diagnostic or treatment related parameters.

The terms energy, energy application, radiation and irradiation, as contemplated within the scope of this patent application may, in some instances be interpreted widely, and originate from different kinds of energy sources, for example, electromagnetic, thermal, particle, or acoustic radiation, and may be ionizing or non-ionizing. It may further include the application of magnetism or electricity, for example, in the form of magnetic or electrical fields, or currents that may be pulsed or alternating. By way of example, the energy source can provide optical radiation or radiofrequency radiation, among other types of energy.

The energy conducting element incorporated within the implant may be demarcated with markers allowing for alignment with the pairing device to ascertain functional engagement. As such, the system may use different combinations of markers (e.g. radiopaque fillers, metal rings, and the like) image guidance, navigation, robotic assistance, and image methodologies, such that a surgeon or operator:
a) may visualize and guide the devices into position (e.g. using x-ray based image-guidance), or
b) has knowledge that devices are physically aligned with each other to achieve functional association (pairing) or disassociation (unpairing). Alternatively, alignment may be done with the naked eye visually by the operator utilizing graduation marks on the devices, or making use of mechanical stoppers.

The implant or other parts of the system according to embodiments of the disclosure may further be made of an energy resistant or energy transmitting or energy diffusing or energy reflecting or energy refracting material. The implant's energy resistant properties may prevent the implant from being substantially damaged by energy exposure or its function being compromised. At least in part, the presence of diffusing properties ensures that energy such as photons may originate from within the vessel lumen, diffusely travel through at least a portion of the implant and vessel wall, and be substantially diffusely emitted, toward the target tissues. The transmissive properties may, for example, be realized by selection of materials with high energy transmittance. In the case of optical energy, materials such as transparent or translucent stent strut or graft material, or thin stent struts may be used. Furthermore, the implant design may be optimized to minimize the struts scaffold surface area vs. the tubular surface area. Some examples of such materials can include, without limitation, metals, alloys, plastics, and polymers. In some embodiments, a stent according to the present teachings can be formed using a metallic mesh, where mesh is at least partially surrounded by a radiation diffusing element (e.g., a polymeric element that can scatter the radiation incident thereon). At least a portion of the radiation passing through the openings of the mesh will undergo diffusion by the radiation diffusing element such that at least some of the radiation is redirected to regions of the external tissue where the radiation intensity, in the absence of the radiation diffusing element, would have been too low, due to a shadowing effect caused by the metallic portions of the stent. Further, by redirecting some of the radiation that would have otherwise resulted in regions of excessive light intensity in the illuminated tissue (herein referred to as “hot spots”), the light diffusing element can improve the uniformity of the light intensity in the illuminated regions.

The diffusing properties may for example be realized by roughening smooth surfaces or incorporating scattering materials, e.g., within the body of the stent. Examples include, but are not limited to, metal nanoparticles, metal oxides, tin oxide, aluminum oxide, titanium oxide, chromium oxide, polylactic acid, polyurethane, polyester, polyethylene terephthalate, polytetrafluoroethylene and ultra-high molecular weight polyethylene, porous materials, nanofibers, electrospun materials, woven materials, knitted materials, expanded materials, sintered materials etc.

The reflective properties may for example be realized by use of metal coatings and other photon reflecting materials.

In some embodiments, the energy conducting element may be configured to exhibit a predefined energy transfer function, which can reduce, and preferably minimize, undesired inhomogeneities in the radiation intensity in the illuminated region (e.g., to reduce and preferably minimize hot and cold spots). In addition to, or instead of, reducing intensity inhomogeneities, the radiation diffusing element may be customized to exhibit a radiation transfer function that is configured to provide an efficient illumination of the anatomy of the target tissues.

In some embodiments, the radiation diffusing element capable of modifying the intensity profile of the radiation (or other energy type) illuminating the external tissue (e.g., via a designed energy transfer function) is realized by use of materials having an energy absorption (possibly approaching zero) which is less than the energy absorption of the surrounding biological tissues, or whose scattering properties (possibly approaching zero) are less than the scattering property of the surrounding biological tissues, for example in the form of a mechanical support structure such as a tube made of polymer material, or in the form of a mechanical scaffold with a covering graft material, wherein the polymer material of the tube or the graft material of the mechanical scaffold has a lower energy absorption or lower scattering properties, compared to the surrounding biological tissues, in order to render the predefined energy transfer function more homogeneous with minimized hot and cold spots. Examples of suitable polymers include polyurethane, polyester, and polyethylene terephthalate.

In another embodiment, a design element (alternatively called an energy transferring element) capable of modifying the energy transfer function is realized by use of materials having an energy absorption which is less than the energy absorption of the surrounding biological tissues, and whose scattering properties are higher than the scattering property of the surrounding biological tissues. In some embodiment such design element may include a mechanical support structure like a metal scaffold with struts, which is in principle hindering energy conduction, but which is provided with a surrounding graft material. In some embodiments the graft material may have a lower energy absorption, but higher scattering properties, compared to the surrounding biological tissues, in order to render the predefined energy transfer function more homogeneous with minimized hot and cold spots.

In yet some other embodiments of the disclosure, the design element capable of modifying the energy transfer function is realized by use of materials where a surrounding graft material may have a thickness that is larger than the scattering free path length of the graft material, so that for each average photon, at least one scattering event may occur before it is emitted by the implant. This thickness may be in the order of magnitude of circa 10 μm.

Furthermore, in some embodiments, the design element capable of modifying the energy transfer function may be realized by use of materials in which the thickness of the mechanical support structure, e.g. the diameter of the metal struts, may be smaller than the thickness of the surrounding graft. In some embodiments, the design element capable of modifying the energy transfer function may be realized by use of materials in which the stent strut thickness is less than the scattering mean free path length of the surrounding graft material.

In yet another embodiment, the design element capable of modifying the energy transfer function may be realized by the use of a mechanical support structure, like a metal scaffold with struts, which can hinder energy conduction, but wherein the energy conducting element is able to be repositioned within a bodily vessel or lumen of a patient, in order to render the predefined energy transfer function more homogeneous with minimized hot and cold spots.

In some other embodiments, the design element capable of modifying the energy transfer function is realized by the use of materials of a mechanical support structure like a metal scaffold with struts, which can hinder energy conduction, and wherein the energy conducting element is at least partially made of a material, e.g. a graft material, with a high energy conduction, wherein the effective surface area of the energy hindering material in relation to the effective surface area of the material with a high energy conduction may be at least 1:5 for low:high energy conducting materials. As an example, alloys such as steel and nitinol can be considered as energy hindering materials, whereas polymers such as polyurethane and polytetrafluoroethylene are energy transferring materials, in case the applied energy is in the form of light.

Further disclosed is that, as a part of the aforementioned system according to the disclosure, in some embodiments at least one pairing device:
a) is placeable endoluminally within the implant and comprises at least an energy conducting element with a predefined energy transfer function, or
b) has docking capability with the implant with the ability of being functionally engageable with or releasable from energy conducting element, or
c) is demarcated with markers allowing for the alignment with the implant to ascertain functional engagement with the energy conducting element, or
d) has at least one compressible and expandable member such that it may be juxtaposed against the implant providing a contact surface and is used to displace the media within the vessel avoiding energy absorption by the media or to provide a predefined distance between the system components and vessel or conduit wall, or
e) comprises at least one hollow canal/lumen, for example, for it to be substantially non-occluding and allow bodily fluids and substances to flow through such hollow canal/lumen; or for at least one energy-emitting element to be placed within the hollow canal/lumen; or for at least one sensory probe, detecting, or imaging device to be placed within the hollow canal/lumen.

In various embodiments, one or more of the characteristics disclosed herein with regard to the implant may also apply for the pairing device(s).

Furthermore, in some embodiments a pairing device may be an implant delivery device, usable for the deployment of the stent-like implant in a vessel or conduit of the patient.

In order to anchor the implant at a desired site, the stent-like implant may be converted from a compressed state into an expanded state via an appropriate steering of the implant delivery device.

On the other hand, the implant or such a pairing device may have at least one canal/lumen for placement of a separate energy emitter, allowing for easy and fast placement of the stent-like tubular implant and of an energy emitter in one session, without the need for multiple device exchanges, with similarities to currently available pre-loaded stent systems.

In some embodiments, the system may further comprise a pairing device being an energy-emitting element, having a predefined energy emission function profile. Such profile may for example be circular, elliptical, or shaped as a ‘top hat’.

A system according to the disclosure may further comprise a pairing device being a sensory, detecting, or imaging device. The sensory or imaging probe may include e.g. optical imaging, intravascular ultrasound, optical coherence tomography, endomicroscopy, etc.; and could be used for measurement of treatment related parameters, for example, but not limited to:
a) Tissue morphology and tissue function,
b) Temperature,
c) Tissue oxygenation,
d) Fluid dynamics—Hemodynamics e.g. Blood volume and flow,
e) Measurement of irradiation and dose related parameters,
f) Substance, drug, or photosensitizer concentration,
g) Tissue properties (e.g. optical absorption, scattering, reflectance),
h) Fluorescence (e.g. exogenous and exogenous, to assess the tubular implant and/or vessel of interest over its longitudinal access as to detect the optimal target area for therapy).

According to a further embodiment, the system according to the disclosure may comprise an intra-arterial or endovascular substance delivery device for a local substance delivery, e.g., a contrast agent, drug, fluids, particles, beads, photosensitizer, radiosensitizer, energy absorber, catalyst, etc.

According to the disclosure, the stent-like implant may be constructed from materials that are flexible and have shape memory for it to be closed by an external compression force and re-open when such compression force is removed, without compromising the mechanical integrity of the implant. A surgeon may utilize an atraumatic vascular clamp to temporarily close the vessel with implant in the case of an inadvertent vascular rupture, thus allowing for surgical vessel repair and re-opening of the stent-like implant after repair. Some embodiments contain mechanisms and methods to temporarily induce a degree of localized hypoxia within the wall of the vessel or biological conduit, but not in the surrounding tissues. For example, by temporarily exerting an increased radially outward pressure on the vessel or conduit wall in order to, for example, temporarily compress the vaso vasorum to render the vessel- or conduit-wall less susceptible to an energy induced damage that is oxygen dependent.

Some embodiments may utilize a 2D or 3D sensing, probing, or imaging system (e.g., a computed tomography (CT) imaging system, a magnetic resonance imaging (MRI) system, an Intravascular ultrasound system (IVUS), an optical coherence tomography (OCT) system, a photoacoustic imaging system,) a microscopy system and spectroscopy system. For example, a confocal laser endomicroscopy system may be deployed, where a small probe is placed against the lumen wall to provide in vivo visualization of the cellular structures of the lumen wall, to provide endoluminal or exoluminal data. For example, used together with a software application for the customization i) of the physical dimensions of the system components and ii) as well as their energy transfer functions and emission profiles, they may be used to tailor and customize the implant or its pre-defined energy transmission function to the individual dimensions of a patient's vessel, conduit or lumen and target tissues. For example, 2D and 3D imaging systems may be used to customize or tailor or fit the physical dimensions of the implant to the dimension of the patient's vessel or to customize the energy emission profile to fit the anatomy of the target tissues. It may further be used for a guidance or assessment of the tissue response. For example, if the tumor has a varying thickness along the length of the vessel, the energy transferring element can be configured to have a tailored gradient in its energy transfer function such that the dose delivered is customized to the specific anatomy.

In some embodiments, the sensing, probing, or imaging system may be used determine an appropriate vessel wall protection device or method. In one example, if the endoluminal probing sensing or imaging data (e.g. IVUS and OCT) demonstrates an apparently abnormal extracellular matrix (ECM) with apparent changes in the thickness of the extracellular matrix or a partial absence thereof, and, if there is apparent invasion of the tumor tissues into the vascular wall, as assessed endoluminally and/or via standard 3D imaging such as CT and MRI, then it may be decided to proceed with energy-based application using a stent-like protection device. In another example, if the endoluminal probing, sensing or imaging data (e.g. IVUS and OCT) demonstrates an apparently normal extracellular matrix (ECM) with no apparent changes its thickness, and, there is no apparent invasion of the tumor tissues into the vascular wall as assessed endoluminally and/or via standard 3D imaged such as CT and MRI, then it may be decided to proceed with energy based application without a stent-like protection device, but rather with temporarily compressing vasa vasorum and/or collagen crosslinking to provide an in situ vascular scaffold.

In some embodiments, the imaging system may be utilized to track a position of the energy-emitting element and/or an endoluminal wall protection device within a vessel. Utilizing an imaging system to track and guide the position and deployment of the energy-emitting element and/or an endoluminal wall protection device may aid in preventing inadvertent vessel damage as the position of the emitting element and/or an endoluminal wall protection device may be adjusted to avoid contacting the vessel wall. In other embodiments, wherein the imaging system includes an IVUS or an OCT, the imaging system may be utilized to inspect structural integrity of the vessel wall. In these embodiments, the imaging system includes a probe capable of assessing the vessel wall and in particular examine its integrity, having a penetration depth in the range of circa 1-10 millimeters and a resolution in the range of several micrometer to several hundreds of micrometers. Providing an imaging system with this resolution may provide information at the anatomic, functional, and molecular level. This information may be used to determine structural integrity of the vessel before the energy-emitting element emits energy. Providing a system that allows a physician to analyze the structural integrity of the vessel before the energy-emitting element emits energy may avoid further damaging the vessel as, if the physician determines the vessel is damaged based on the information provided by the imaging system, the energy-emitting element may be controlled provide less or no energy to the target tissue that may otherwise cause damage to the vessel. The imaging system may also be utilized to provide anatomical information in a macroscopic range (e.g., in the sub-millimeter to several centimeter rang) about the vessel about the target vessel and surrounding tissues. The imaging system may further be utilized while the energy-emitting element is emitting energy to further assess the structural integrity of the vessel.

The imaging system generates image data that includes a region of interest. The region includes the vessel and/or the target tissue. The image data may be displayed by a display of the imaging system. Based on the image data, the system may determine a dose contour map and may overlay the dose contour map on the displayed image. This can be performed in a fashion that is similar to radiotherapy treatment planning, for example, using dose contour maps and tissue damage thresholds superimposed on CT images that provide the anatomical information and electron density of the target tissues. The system may be further configured to determine functional information e.g., on tissue perfusion (e.g., hyperperfusion, hypoperfusion) based on the image data. This functional information may be utilized to visualize and monitor a tissue response to the emitted energy. Furthermore, the image data may be utilized to provide anatomical information that is then subsequently displayed and superimposed with the previously discussed estimated of damage thresholds. These damage thresholds may be estimated based on generic literature values, iterative learning from previous patients and may also be determined in a patient specific manner. The latter, for example, by measuring the tissue optical properties via spectroscopy and in doing so more accurately estimating the dose contours and thresholds.

The system according to another embodiment of the disclosure may utilize a treatment planning method. The treatment planning method may plan, guide, and/or monitor progression of a treatment that utilizes aspects of the disclosure to facilitate safe, accurate and controlled endovascular energy delivery. In one embodiment such treatment planning can for example be carried out using Monte Carlo dose simulations to estimate the dose delivered to the target tissues and determine the relevant threshold doses and estimated tissue response.

The system according to another embodiment of the disclosure may be adapted accordingly so that at least one of the implants or pairing device(s) may be coupled via a tubular construct (e.g. a catheter) to an extra-corporeal unit (e.g. an energy source, a sensory probe read-out, analysis device, treatment planning and monitoring system) forming a comprehensive treatment, planning, guiding or monitoring system. In some embodiment the system contains a cooling mechanism that may be, for example, active or passive. In some embodiments, predefined energy transfer and energy emission functions (e.g., causing the emitted energy to have an elliptical, cylindrical or customized shape) of the system and its components enable for an accurate estimation of an energy dose arriving at the target tissues, allowing for a prediction of the treatment effect and for a treatment planning analogous to radiotherapy.

Some embodiments further provide a method for the transfer of energy emitted within the lumen of a bodily vessel or conduit directed outwards through the wall of the vessel or conduit (or in the reverse direction) for the application of energy to target tissues in the surroundings of the bodily vessel or conduit, used for immunomodulation or pathogen inactivation or causing cell death of tumor tissues surrounding the vessel or conduit, while protecting the vessel or conduit and leaving the structural integrity of the vessel or conduit intact, by use of a system that may comprise at least one of the following components:
a) a stent-graft-like implant that may be tubular in shape and may comprise at least in part, an energy conducting element with a predefined energy transfer function, and
b) the implant having docking capability with at least one separate pairing device with the ability of being functionally engageable with or releasable from the energy conducting element.

In some embodiments, the method may be used for the purpose of modulating the immune system to optimize immunotherapeutic approaches, especially for optimizing antigen presentation and priming of the immune system via the endoluminal route utilizing endoluminal energy therapy, for example, combinatorial energy-based ablation and immunotherapy, photodynamic therapy, and/or photoimmunotherapy.

In some embodiments, the method may be utilized to create a circumferential tumor free margin around a blood vessel, while leaving the vessel wall substantially unharmed. The method may create this margin by damaging or killing some or all of the target tissues located at a margin of the blood vessel, which may be target issues that are in contact with the wall of the blood vessel or are within a short distance from that wall. In different embodiments, the short distance may be determined by the required thickness of the tumor free margin. This required thickness may be determined such that it effectively isolates the vessel from affixed surrounding tissues and enables surgical removal of the tumor without damaging the blood vessel. In some embodiments, this thickness may be in the order of several millimeters. In some embodiments, creating the margin may result in downstaging pancreatic cancer patients with locally advanced disease such that they become eligible for surgery.

In some embodiments, the disease free margin is a tumor free margin. In particular, the disease free margin may be a circumferential margin around the biological vessel. In some embodiments, the disease free margin, preferably tumor free margin, extends at least around 1 mm, at least around 2 mm, at least around 3 mm, at least around 4 mm, at least around 5 mm, at least around 10 mm, at least around 15 mm, at least around 20 mm, at least around 25 mm, at least around 30 mm, at least around 35 mm, at least around 40 mm, at least around 45 mm, or at least around 50 mm from the center of the biological vessel. Preferably, the disease free margin extends between around 1 mm to around 2 mm, between around 2 mm to around 4 mm, between around 4 mm to around 6 mm, between around 6 mm to around 8 mm, between around 8 mm to around 10 mm, between around 10 mm to around 12 mm, between around 12 mm to around 14 mm, between around 5 mm to around 30 mm, around 5 mm to around 25 mm, around 5 mm to around 20 mm, around 10 mm to around 30 mm, around 10 mm to around 25 mm, or around 10 mm to around 20 mm from the center of the biological vessel. Preferably, the disease free margin comprises the lumen of the biological vessel, the vessel wall and the target tissue directly contacting the biological vessel.

According to a further embodiment, the method according to the disclosure may be used for treatment of patients with malignant obstructions in vessels or conduits, for example, in the bile duct or blood vessels.

In yet another embodiment, the method according to the disclosure may be used for pathogen inactivation, in patients with urinary tract infections.

Moreover, in some embodiments, in a method for endoluminal and non-endoluminal photodynamic therapy, a photosensitizer may be administered intra-arterially to reduce cutaneous photosensitivity. Furthermore, disclosed are energy sensitive substances or compositions of matter that can be activated with energy for partial or fully circumferential ablation of a diseased tissue surrounding a lumen or vessel where the energy is delivered via the endoluminal/endovascular route and the structural integrity of the lumen of vessel is largely maintained.

In some embodiments, the energy sensitive substances may include known substances employed for novel uses. The novel uses may include, for example, the disclosed techniques of partly or fully circumferential ablation of a diseased tissue surrounding a lumen or vessel, where the energy may be delivered via the endoluminal/endovascular route and the structural integrity of the lumen of vessel is largely maintained. The techniques may be utilized for downstaging cancer patients and allowing for the isolation of vessels from their affixed surrounding tissues to facilitate surgical resection with tumor free margins around such vessels.

In one embodiment such substances may include a member of at least one of the following groups:
a) a photosensitizer activated by energy from photons;
b) a photocatalyst activated by energy from photons;
c) a radiosensitizer activated by energy from radiation;
d) magnetic particles activated by magnetic energy;
e) particles activated by energy from photons; and
f) a sonosensitizer activated by acoustic energy.

In some embodiments, the energy-activatable substance is an energy-activatable substance capable of being activated outside of a blood vessel by energy emitted from an energy-emitting element that is located in the lumen of the blood vessel.

In some embodiments, the energy-activatable substances may be used in conjunction with chemotherapeutic agents, which may be taken up by tissues that have been exposed to energy and as a result have become more susceptible to substance/drug uptake (e.g. tissues and cells may have become more porous via electroporation, electrochemotherapy and the like).

In some embodiments, the photosensitizer substances may include compositions of matter that upon excitation with photons culminate in photochemical reactions (e.g. type I and type II). Examples are porphyrins, chlorins, bacteriochlorins, phthalocyanines, transition metal complexes and the like, including derivatives and modifications thereof, as well as derived metal complexes, specifically: benzoporphyrin derivatives, meta(tetrahydroxyphenyl)chlorin, porfimer sodium, protoporphyrin IX and aspartyl chlorin.

These embodiments include but are not limited to zinc-, aluminum- and silicon-phthalocyanines, tri- or tetrasulfonated phthalocyanine, monosulfonated aluminum chlorophthalocyanine, aluminum cholorophthalocyanine, zinc(II)-phthalocyanine, silicon-phthalocyanine derivative (C₇₄H₉₆N₁₂Na₄O₂₇S₆Si₃), porfimer sodium, HPPH 2-[1-hexyloxyethyl]-2-devinyl pyropheophorbide-a, 3-(1′-hexyloxyethyl)pyropheophorbide-a, protoporphyrin-IX, hematoporphyrin, hematoporphyrin derivative, talaporfin, talaporfin hydrochloride, talaporfin sodium, chlorin e6, chlorin e6 sodium salt, chlorin e6 trisodium sal, WST09, padoporfin, Pd-bacteriopheophorbide, WST11, padeliporfin, palladium bacteriopheophorbide monolysotaurine, verteporfin, benzoporphyrin derivative monoacid ring A, BPD-MA, temoporfin, mTHPC, (5,10,15,20-Tetra(m-hydroxyphenyl)chlorin), hypericin, rose Bengal, thiazine, methylene blue, toluidine blue, TLD1433 or any pharmaceutically acceptable derivative thereof selected from pharmaceutically acceptable salts, esters and amides.

These may be unspecific, or targeted by conjugation to a targeting moiety such as antibodies, peptides etc. and may be part of a lipid or multi-functional nanoparticle system. Within the scope of this disclosure are “energy sensitive-” or “energy-activatable-substances” being any substances, compositions of matter or particles that can absorb energy and lead to a biological effect. In one embodiment the used substance can transfer absorbed energy into heat. For example, using dye or gold nanoparticles, taken up by a target tissue and which absorb energy in the form of light, alternating electrical field or currents (radiofrequency and microwave ablation) and subsequently can transfer this applied energy into heat. In doing so, the target tissues with dye or gold nanoparticles are heated up more so than non-target tissues without, leading to the selective destruction of target tissues. In yet another embodiment, the “energy sensitive-” or “energy-activatable-substance” may be temperature sensitive, carry a load and release such load upon energy activation. One example is a temperature sensitive liposome carrying a toxic chemotherapeutic load—which upon absorbing energy and reaching a certain temperature can release its toxic load to selectively destroy tissues.

In some embodiments, energy sensitive substances may be used to downstage patients with certain diseases, facilitating surgical resection with disease-free margins around the lumen or vessel e.g. of pancreatic cancer patient.

Some embodiments disclose energy sensitive substances in combination with a stent-like device, which may aim to provide structural support to the lumen wall.

In some embodiments, an energy sensitive substance may be used with a stent-like device having a predefined energy transfer function, aiming for a controlled dose of energy to be delivered to the target tissues.

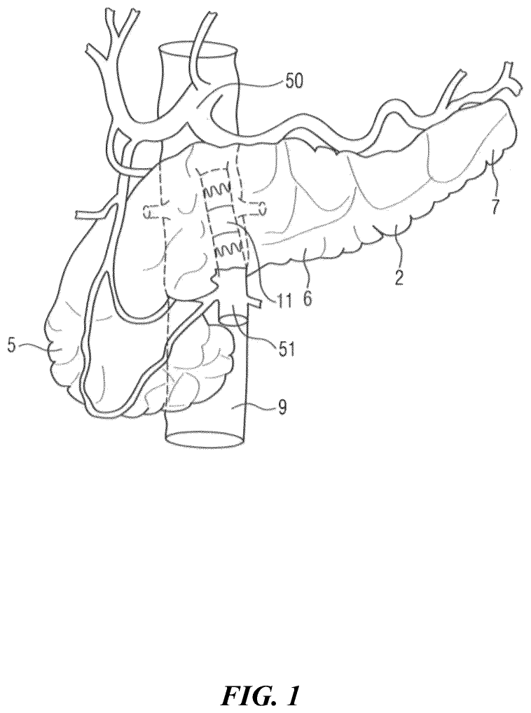

In the following, the disclosure shall be outlined by an exemplary treatment of pancreatic cancer. FIG. 1 is a perspective view of a human pancreas and adjacent arteries with a stent-like implant 11 according to an embodiment of the disclosure deployed in the superior mesenteric artery (SMA) 51, or arteria mesenterica superior.

Surgical interventions to the pancreas are generally considered challenging, due to the complexity of the anatomy.

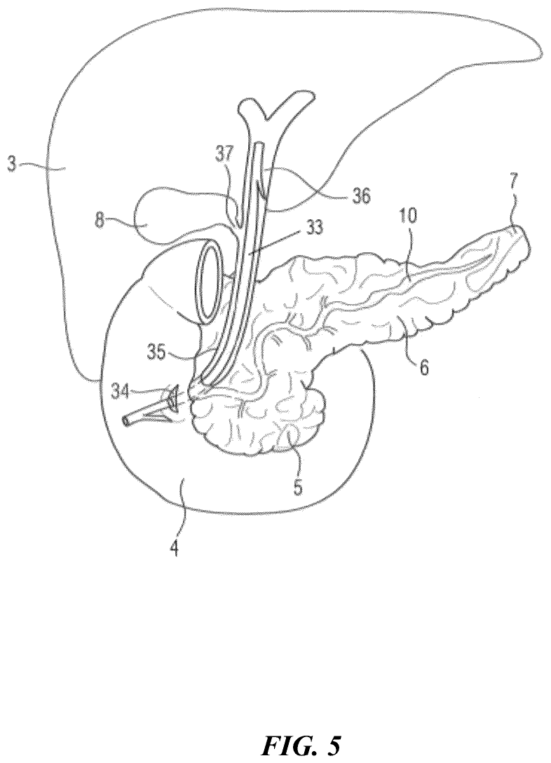

As shown in FIGS. 1 and 5, the pancreas 2 is a gland in the retroperitoneum, beneath the liver 3, left of the duodenum 4. The pancreas 2 is subdivided into a head or caput pancreatis 5, a body or corpus pancreatis 6, and a tail or cauda pancreatis 7. The cauda pancreatis 7 contacts the spleen and kidney on the left side of the human body. The main bile duct coming from the liver 3 and the gallbladder 8, traverses the caput pancreatis 5 on the right side of the human body. The excretory ducts of the pancreas 2, especially the ductus pancreaticus 10 and the ductus santorini, pass through the entire pancreas 2.

Further, FIG. 1 shows that the aorta 9 and several mesenteric arteries, especially the truncus coeliacus 50, the arteria mesenterica superior 51, arteria mesenterica inferior and arteria lienalis, are in close contact to the pancreas 2, as well as the vena cava inferior and the vena portae. The pancreas 2 is supplied with blood via the truncus coeliacus 50 and via the arteria mesenterica superior 51, and sometimes from the arteria hepatica propria. The de-oxygenated blood leaves the pancreas 2 through the vena mesenterica superior and the vena lienalis.

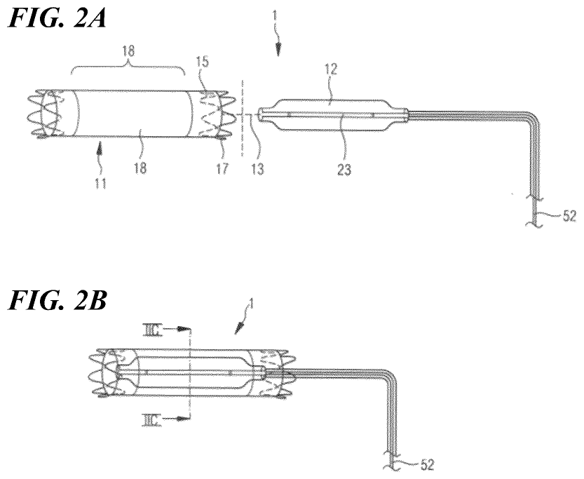

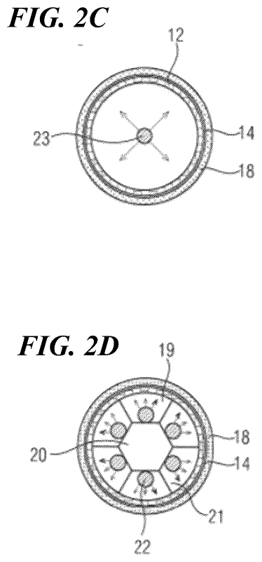

FIGS. 2A-2D show a system 1 for applying energy to a target tissue and some of its components according to some embodiments.

In particular, FIG. 2A is a perspective exploded view of the system for the transfer of energy emitted within the lumen of a bodily vessel like a blood vessel in the human pancreas. In FIG. 2A. System 1 comprises the stent-like implant and a pairing device, wherein the component devices are unpaired and functionally disengaged.

FIG. 2B is a perspective view of the system of FIG. 2A with its component devices docked together in coaxial alignment, and therefore being paired and functionally engaged.

FIG. 2C is a cross-section view of the system of FIG. 2B along line IIC-IIC.

FIG. 2D is a cross-section view similar to FIG. 2C of a system according to another embodiment of the disclosure.

System 1 comprises a stent-like implant 11. The stent-like implant 11 may further include an energy transferring element 18 and a docking capability (element/structure) which may allow at least one separate pairing device 12 to be docked to the stent-like implant 11. Furthermore, the docking capability may allow engagement between the pairing device and the stent-like implant 11 as well as the ability of a release therefrom.

The stent-like implant 11 may be designed for introduction and placement within a tubular structure of a patient like a blood vessel, for example, within the arteria mesentarica superior 51, shown in FIG. 1; or within an adjoining artery or other vein, and in particular in an area where cancerous tissues or any other pathologies to be treated are located directly on the outside of the blood vessel in question.

Before the treatment, a substance may be administered to the patient, selected in such a way that it accumulates to a particular extent in or on diseased tissue, such as cancerous tissue. The substance may be chosen such that it is energy sensitive and may develop its treatment effect, e.g. cell-killing effect, through a physical stimulation, for example, after stimulation with energy, e.g., by light or thermal radiation or some other form of radiation.

In system 1, the stent-like implant 11 has a tubular or cylindrical shape with a longitudinal axis 13. In order to provide such tubular or cylindrical shape, the stent-like implant 11 may comprise a supporting structure 14, for example in the form of a tube or a cylinder jacket, as may be seen from FIGS. 2C and 2D.

Referring now to FIGS. 2A and 2B, such supporting structure 14 may be a braid, fabric, or mesh 15 made of individual threads or wires, such as metal wires, and may have a tubular geometry.

In the embodiment shown in FIGS. 2A-2B, the metal wires are each be bent in a wave shape, and each such wave shaped elements are closed into a ring-shaped element. Several such ring-shaped closed elements, each made of a metal wire bent in a wave-shaped manner, may be arranged one behind the other or next to one another in the longitudinal direction 13 of the stent-like implant 11 and connected to one another, for example glued or soldered, in order to form the mesh 15 of the supporting structure 14. Mesh-like supporting structures 14 may also be manufactured using for example laser cutting of a metal cylinder or any other suitable manufacturing technique.

The supporting structure 14 of the stent-like implant 11 may, in some embodiments, be covered or surrounded on its outside or inside by the energy transferring elements 18. In some embodiments, the energy transferring element 18 may be made of a light transmitting and diffusing material, or the scaffolding material itself may be made from a light transmitting and diffusing material.

FIGS. 2A and 2C show one kind of a pairing device 12 according to some embodiments. The pairing device 12 may be used as an implant delivery device. Such an implant delivery device may be used as a vehicle to transport the stent-like implant 11 to a desired position within a vessel or lumen of a patient and to deploy it there. For this purpose, a distal portion of the implant delivery device may fit into the tubular stent-like implant 11, for example, in a compressed state of the stent-like implant 11.

Furthermore, in some embodiments, the implant delivery device may have an inner lumen and thus may be threaded over a previously placed mandarin/guidewire. Further, the implant delivery device may be pushed forward along such mandrin/guidewire to the area to be treated, with the compressed stent-like implant 11 placed piggyback on the distal portion of the implant delivery device.

When the distal portion of the implant delivery device has reached the desired location, the stent-like implant 11 may be expanded and deployed at the target area. The mandrin/guidewire may be used to introduce the implant delivery device and may be removed if necessary when the distal portion of implant delivery device is in the desired position. After removal of the mandrin or guidewire, it may be replaced by an energy-emitting device and the target tissues may be irradiated.

FIG. 2D further shows the distal portion of the implant delivery device according to some embodiments. The distal portion may have a plurality of chambers 21 in its interior. The chambers 21 may be subjected to extracorporeal pressure, for example via a fluid, such as a liquid or a gas such as air. Such an internal overpressure causes the chambers 21 to swell and thereby enlarge their cross-section, especially radially outwards.

These chambers 21 may be used to deploy the stent-like implant 11 at a target site. Therefore, as may be seen in FIG. 2B, if the implant delivery device being placed inside of the stent-like implant 11 has reached the target area, then the radially outer regions of the hollow chambers 21 or a balloon surrounding them may be expanded and thereby come into contact with the supporting framework or scaffold 14 from the inside and press outward. When the pressure increases further, the scaffold 14 may be deformed and expanded, and then juxtaposed against the inner wall of the relevant blood vessel 51 or bodily vessel from the inside. In the expanded state, the supporting framework or scaffold 14 of the stent-like implant 11 may be frictionally pressed against the inner wall of the relevant vessel 51 and anchored in place. In the process, the mesh 15 of metal wires may be deformed, thereby causing the wave-shaped, ring-shaped closed elements 17 to stretch, so that the curvature of the waves form decreases. During this process the media within the vessel, e.g. blood, may be pushed away and in doing so minimize the chances that such a media would hinder energy transmission. For example, the presence of blood would likely hinder the transmission of light in the case of PDT.

In the embodiment shown in FIG. 2D, the distal portion of the implant delivery device may be configured to include light-emitting elements 22. In this case, the pairing device 12 may have the structure of a light emitting device, too. In this case, after the anchoring of the stent-like implant 11, the light-emitting elements 22 arranged within the pairing device 12 may be activated. The light-emitting elements 22 may be optical fibers 52, which extend into the region of the chambers 21 in the distal portion of the implant delivery device, and which emit their radiation radially outwards. This may be achieved, for example, by roughening the relevant surface areas of the outer surface of the optical fibers 52, so that light emerges to the outside.

The light emitted in this way may transmit through the outer covering or wall of the chambers 21, through the energy transferring element 18 of the stent-like implant 11 and through the adjoining vascular wall into the target tissue surrounding the artery. The light may activate there the accumulated substance such as a PDT photosensitizer substance within the target area, which in turn may treat (e.g. kill) the cells concerned.

In some embodiments, the pairing device 12 functions as an implant delivery device used to deliver the stent-like implant 11, and does not have any light-emitting elements 22 and is removed after the placement of the stent-like implant 11. The removed pairing device 12 may be replaced by a second pairing device 12, which is provided with at least one light-emitting element 23. After it is placed concentrically within the stent-like implant 11, the light-emitting element 23 may be activated extracorporeally, for example by irradiating light from a laser. This light then may emerge at the roughened end areas of the optical fiber 52 and then reach the diseased tissue or a substance absorbed therein (which is herein referred to as an substance e.g. a photosensitizer), which, when activated by energy (e.g., light or radiation of a specified wavelength), may damage (e.g., kill) the diseased tissue.

The wavelength of the emitted light may be selected to allow activation of the previously administered active substance in such a way that the latter reacts particularly sensitively to the irradiated wavelength and is put into an activated state that can facilitate damaging and preferably killing the diseased tissue. In many cases, the radiation may be red to infrared light (e.g., with a wavelength in a range of about 600 nm to about 1500 nm) because photons of such wavelengths may be absorbed comparatively little by blood vessels or tissues, and may therefore be able to penetrate the target tissue up to a depth of several millimeters. For instance, the radiation emitted by the energy-emitting element, preferably a light-emitting element, may be between about 500 nm and about 900 nm, between about 500 nm and about 800 nm, between about 500 nm and about 700 nm, between about 600 nm and about 900 nm, or between about 600 nm and about 800 nm. The light may also reach deeper lying tissue layers, which are further away from the blood vessel concerned, while still carrying enough energy to activate the active substance at those distant locations.

As may be seen from FIG. 2D, the cavity or lumen 20 may be open; this not only may favor insertion on a mandrin or guidewire and exchange with an energy-emitting element, but also may have the further effect that the relevant blood vessel is not closed as soon as the pairing device 19 is placed there. Rather, the blood may continue to flow through this central cavity or lumen 20.

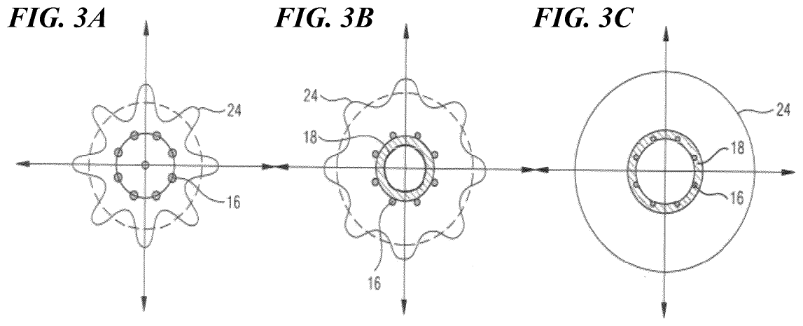

Further characteristics of some embodiments of the disclosure may be seen in FIGS. 3A-3C. More specifically, FIGS. 3A-3C show some components of the device and the corresponding energy distribution profile curves 24 according to three different embodiments, further described below. The energy distribution profile curves indicate the azimuthal magnitude of the emitted light intensity, i.e., the magnitude of the emitted light intensity as a function of the circumferential angle.

In particular, FIG. 2A is an angular energy distribution diagram representing the energy transfer function of a system similar to FIG. 2B. In FIG. 3A, the diagram shows the energy emitted in the cross-sectional plane according to line IIC-IIC of FIG. 2B in a direction radially outwards through a stent-like implant with a scaffolding structure. The scaffolding structure comprises thick struts or wires, which may hinder the energy transmission and thereby cause undesirable inhomogeneities in the emission profile.

More specifically, FIG. 3A shows the relationships in the arrangement according to FIGS. 2A and 2C: A light-emitting element 23 arranged along the longitudinal axis 13 is surrounded by a network 15 of metal wires 16. These metal wires 16 are shown in FIG. 3A by thick dots, corresponding to a section across a wavy meandering metal wire 16.

Since the metal wires 16 may block light and other radiation or at least absorb, reflect and/or scatter a portion of the light passing therethrough, they each may cause a shadowing effect by blocking or reducing the amount light that reaches certain locations of the external tissue at least partially surrounding a vessel. For example, they can exhibit an energy transfer function as depicted in FIG. 3A, where the curve 24 is intended to represent an angular distribution of the energy transfer emitted outwards around the light-emitting element 23 beyond the mesh 15 of metal wires 16. In FIG. 3A, the curve 24 shows the energy intensity increasing as a function of increasing distance of the curve from the center, illustrating variation of the intensity around the light-emitting element. FIG. 3A shows that the light intensity reaches a minimum value in locations (regions) behind a metal wire 16 and reaches a maximum value in locations positioned between neighboring wires 16. This may be caused by a graft material having insufficient diffusing properties and a high energy absorption and/or stent struts that are overly thick and hinder energy transmission, thus causing undesirable inhomogeneities in the emission profile. In addition, the shadowing effect may cause a low overall energy transfer.

FIG. 3B is an angular energy distribution diagram similar to FIG. 3A of a system according to another embodiment of the disclosure. In this embodiment, the stent-like implant 11 comprises an energy transferring element 18 with a graft or covering made of an energy diffusing material with medium energy absorbing properties situated radially inside of a scaffolding structure in order to minimize undesirable inhomogeneities in the energy transfer function.

In this embodiment, the energy transferring element 18 aims to further optimize the energy transfer function. Here, the supporting framework structure 14 may be a mesh of metal wires 16 with a medium strut or fiber thickness (e.g., a thickness in a range of about 10-1000 μm). This supporting framework structure 14 may be surrounded on its inside by a layer of the energy transferring element 18. The element 18 may be a medium that can cause diffusion of light as the light passes through it with substantially no, low, or medium light absorption. For example, in some embodiments, the energy conducting element 18 can absorb less than about 30%, or less than about 20%, or less than about 10%, or less than about 5% of the light passing therethrough while causing diffusion (redirection of some rays) as the light propagates through it. Some examples of suitable materials include, without limitation, polymers like polyurethane, polyester, and polyethylene terephthalate with incorporated in them metal oxides like tin oxide, aluminum oxide, titanium oxide, chromium oxide and the like.

Due to the diffuse effect of this material layer of the energy conducting element 18, the light passing through the conducting element can be redirected such that it will have in addition a radial component, an azimuthal component that would direct the light rays to illuminate the areas radially directly behind the shadowing metal wires 16, thereby reducing the shadowing effect. In addition, such redirection of at least some of the light rays can reduce the intensity in regions where the light intensity exceeds a desired level (“hot spots”), thereby reducing the inhomogeneity of the light intensity.

The setup shown in FIG. 3B, may increase the uniformity of the energy distribution of the transmitted light at the one or more target regions or reduce the shadowing effects of various components of the device at the one or more target regions. In particular, as also shown in FIG. 3B, the amplitude of the fluctuations in curve 24 are lower than the amplitude of those fluctuations in FIG. 3A. Therefore, in this embodiment, some of the undesirable inhomogeneities in the energy transfer function may be reduced. In some embodiments, reducing the inhomogeneities in the energy transfer function may include increasing the homogeneity of the energy transfer function or increasing the uniformity of the energy distribution in the azimuthal direction at the one or more target regions. In some embodiments, increasing the homogeneity of the energy transfer function may include reducing the shadowing effect caused by one or more components of the device, such as the metal wires 16, at the one or more target regions.

A further improved embodiment of the disclosure is shown in FIG. 3C. FIG. 3C is an angular energy distribution diagram similar to FIG. 3B of a system according to yet another embodiment of the disclosure. In this embodiment, the graft or covering of the energy conducting element is made of a material with higher energy diffusion and lower energy absorbing characteristics, and is situated radially outside of the scaffolding structure in order to further minimize undesirable inhomogeneities in the energy transfer function.

Here, the supporting framework structure 14 of the stent-like implant 11 may be a mesh of metal wires 16 with an even smaller strut or fiber thickness. Furthermore, the stent-like implant 11 according to the embodiment of FIG. 3C may comprise an energy transferring element 18 aiming to make further optimizations to the energy transfer function. In this embodiment, the energy conducting element 18 may comprise a layer of a highly light diffusely light transmissive material. Moreover, element 18 may be located on the outside of the supporting framework structure 14. Further, such graft material may include a material that is highly transparent to light. In addition, the thickness of the stent struts or fibers may be minimized. The stent strut thickness may be ranging from circa 10-1000 μm.

The setup shown in FIG. 3C, may further increase the uniformity of the energy distribution of the transmitted light, e.g., by reducing the shadowing effects of various components of the device. In particular, as seen in FIG. 3C, the differences between the maxima and the minima of the energy transfer may be further reduced and the energy distribution, or more specifically the azimuthal energy distribution, may be made more uniform. Alternatively, in some embodiments, the configuration shown in FIG. 3C, may further reduce the shadowing effect caused by one or more components of the device, such as the wires 16. Moreover, the overall energy transfer in this embodiment may be higher than those of the previous two embodiments.

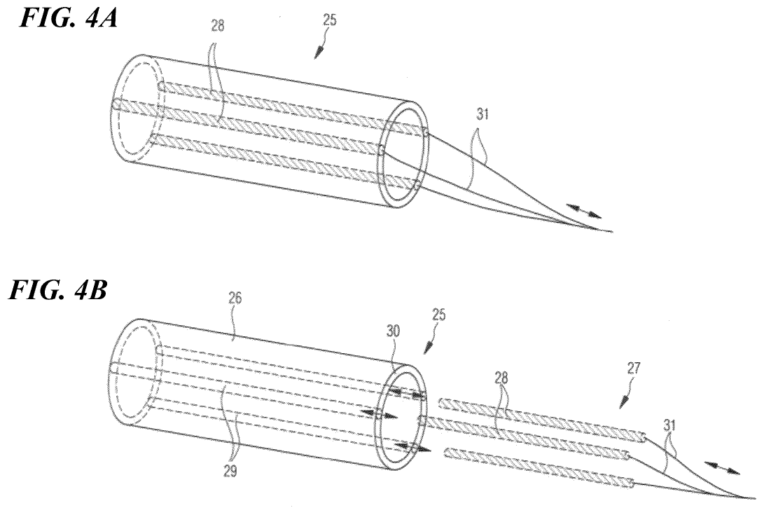

FIGS. 4A and 4B are perspective views of a system 25 according to another embodiment. More specifically, FIG. 4A 4a shows a stent-like implant and energy-emitting devices that are paired and functionally engaged, while FIG. 4B shows those parts being unpaired and functionally disengaged.

In FIGS. 4A and 4B, system 25 comprises a stent-like implant with an energy conducting element 26 and multiple pairing devices 27 in the form of light-emitting elements 28. Elements 28 may include optical fibers 31. Moreover, the system 25 may include a covering 30 for the stent like implant 26. The covering 30 itself may include elongated chambers 29, designed to receive light emitting elements 28.

After the implant 26 has been placed at the site of the target tissue, the light-emitting elements 28 may be activated in order to emit light or other radiation of a suitable wavelength. The emission of light or other radiation may activate the previously administered substance that has accumulated in the target tissues and subsequently trigger its cell-killing effect. By way of example, in some embodiments, a photodynamic therapy drug can be administered to a subject to be preferentially taken up by a tumor. The irradiation of the PDT drug via a radiation source introduced into a vessel in proximity of the tumor in accordance with the present teachings can activate the PDT drug. In some cases, the activated PDT drug can generate radical oxygen species that can cause tumor necrosis.

If necessary, the light-emitting elements 28 may then be removed from their chambers 29 by, for example, being pulled out at their optical fiber cables 31, while the implant 26 remains in place so that it may be reused for subsequent treatments.

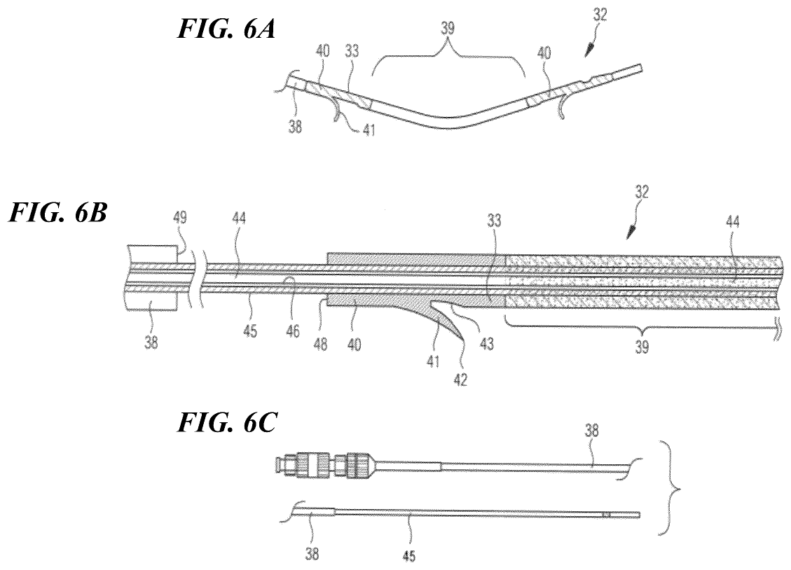

FIG. 5 and FIGS. 6A-6C show another system according to some embodiments. In particular, FIG. 5 is a perspective view of a part of a hepato-pancreato-biliary anatomy with duodenum, pancreas, liver, gallbladder and bile duct and a stent-like implant according to another embodiment described in FIGS. 6A-6C deployed into the bile duct.

FIG. 6A is a plan view of a system 32 for the transfer of energy emitted within the lumen of a bodily vessel like a bile duct as in FIG. 5 according to some embodiments. In FIG. 6A, the component devices are docked together in coaxial alignment, and are being paired and functionally engaged. FIG. 6B is a longitudinal section view of the component devices of the system 32 in a state partially disengaged. And FIG. 6C is a plan view of the pairing device of the system 32 as being disengaged from the stent-like implant.

The system 32 comprises the stent-like implant according to FIG. 5 as well as a pairing device. In particular, the system 32 includes an implant 33. Further, the implant 33 includes a slender cylindrical section 39 having an energy transferring element, which is closed at both ends by a similar cylindrical section 40 made of an opaque or non-energy conducting material.

The system 32 may also be utilized for body lumens other than a blood vessel, such as a bile duct or pancreatic duct.

As may be seen from FIG. 5, the implant 33 may be introduced, for example using ECRP (Endoscopic retrograde cholangiopancreatography) guidance, from the duodenum 4 through the papilla vateri 34 into the main bile duct or ductus choledochus 35. Also, the implant 33 may be introduced through the liver 3 into the ductus hepaticus 36 using percutaneous access.

From there, as required, the immediate vicinity of the main bile duct 35, the liver duct 36, or the gall bladder duct 37 may be accessed in order to treat illnesses such as cancers located there.

These accesses may be made possible by a slenderer design of the implant 33 and the pairing devices 38 and 44 that may be connected to it, as may be seen in FIGS. 6A and 6B.

In some embodiments, while the implant 33 is flexible, it may not be anchored in situ by radial expansion in the same manner described above for some embodiments. Instead, at least one barb-like structure 41 may be provided in the area of each of the two ends 40 of the implant 33. In embodiments that include two barb-like structures 41, these structures may be arranged antiparallel to one another, such that the free ends or tips 42 of the two barb-like structures 41 face one another, for example, in the manner shown in FIG. 6A.

These ends or tips 42 of the barb-like structures 41 may, for example, be pressed inward and pulled into a tube containing a recess 43 provided for this purpose until they are finally placed in the body of the patient to be treated.

After the placement has taken place, the slipped-over tube may be pulled off, whereby the two tips 42 become free and, due to their embossed shape, may protrude outward and get caught in the relevant biliary or liver duct.

A pairing device, comprising a pusher catheter 38 and a guiding catheter 45 with similarities to those shown in FIGS. 6A and 6B may be used to deliver and deploy this implant 33.

This implant delivery devices 38 and 45 may require a previously placed mandrin or guidewire for introduction into the vessel. Accordingly, the implant delivery devices 38 and 45 may have a tube lumen 46 to slide over a mandrin or guidewire. The stent-like implant 33 may be mounted on the implant delivery device 38 and 45, with similarities to a preloaded biliary plastic stent. This combination of the stent-like implant mounted on a delivery device may be used to guide the advancement of the implant delivery device and stent-like device 38 on the mandrin to position the device at the target tissue. The mandrin may be removed after the pairing devices 38 and 45 are in the desired position, and may be replaced by suitable pairing with an energy-emitting element 44. For example, the mandrin may be replaced by an energy-emitting device shown in FIGS. 2A-D comprising one or several light emitting elements 22 or 23 for irradiation of the target tissues. An energy-emitting device may be, for example, an optical fiber 52. As used herein, the light emitting elements may also be referred to as energy-emitting elements.

In some embodiments the energy-emitting element emits energy that is above a cell damage threshold and below a vessel thermal damage threshold. In these embodiments the cell damage threshold relates to an amount of energy needed to cause cell death within a target tissue (e.g., cells within a cancerous tumor) containing the energy-activatable substance and such thresholds are well below thermal thresholds may be determined as a function of tumor type. Furthermore, the vessel thermal damage threshold relates to an amount of energy needed to damage the vessel with heat which is known from previous experiences and is unlikely to occur with irradiances of up to circa 200-600 mW·cm⁻² at wavelengths of 630-700 nm. In other embodiments, the energy-emitting element may be controlled to deliver a total amount of energy to the target tissue. For example, the energy-emitting element may be controlled to deliver 10-1000 Joules·cm⁻², for instance between about 10-100, between about 100-200, between about 200-300, between about 300-400, between about 400-500, between about 500-600, between about 600-700, between about 700-800, between about 800-900, or between about 900-1000 Joules·cm⁻² at the surface of the inner vessel wall, and, directed toward the surrounding target tissues.

In its distal area, the tube 45 of the implant delivery device may be surrounded by an outer tube 38. During the insertion of the implant 33, its proximal end 40 may rest with its end face 48 on the distal end face 49 of the outer tube 38 and then pushed forward by the latter for the deployment of the stent-like implant 33.

The light emerging in the area inside the implant 33 may then penetrate the slim, cylindrical section 39, being the energy transferring element and then reach the target tissues surrounding the relevant bile duct 37 and 36. At those locations, the light may reach the light-activatable substance accumulated there. This energy conducting element 39 may be structurally designed in the same way as the corresponding central section of the stent-shaped implant 33.

After the treatment, the implant delivery device 38 and 45 as well as the energy-emitting device may be removed from the patient's body, while the stent-like implant 33 itself may remain in place and may be used for subsequent treatment.

The preceding may be employed to remodel biological lumen or vessels of patients with certain abnormalities or diseases, preferably used to improve the patency of vessels with compromised blood flow, may be used in combination with chemotherapy, immunotherapy or surgery, and may be used to enable other treatments or to make other treatments more effective.

The above detailed description refers to the accompanying drawings. The same or similar reference numbers may have been used in the drawings or in the description to refer to the same or similar parts. Also, similarly named elements may perform similar functions and may be similarly designed, unless specified otherwise. Details are set forth to provide an understanding of the exemplary embodiments. Embodiments, e.g., alternative embodiments, may be practiced without some of these details. In other instances, well known techniques, procedures, and components have not been described in detail to avoid obscuring the described embodiments.

The foregoing description of the embodiments has been presented for purposes of illustration only. It is not exhaustive and does not limit the embodiments to the precise form disclosed. While several exemplary embodiments and features are described, modifications, adaptations, and other implementations may be possible, without departing from the spirit and scope of the embodiments. Accordingly, unless explicitly stated otherwise, the descriptions relate to one or more embodiments and should not be construed to limit the embodiments as a whole. This is true regardless of whether or not the disclosure states that a feature is related to “a,” “the,” “one,” “one or more,” “some,” or “various” embodiments. As used herein, the singular forms “a,” “an,” and “the” may include the plural forms unless the context clearly dictates otherwise. Further, the term “coupled” does not exclude the presence of intermediate elements between the coupled items. Also, stating that a feature may exist indicates that the feature may exist in one or more embodiments.

In this disclosure, the terms “include,” “comprise,” “contain,” and “have,” when used after a set or a system, mean an open inclusion and do not exclude addition of other, non-enumerated, members to the set or to the system. Further, unless stated otherwise or deducted otherwise from the context, the conjunction “or,” if used, is not exclusive, but is instead inclusive to mean and/or. Moreover, if these terms are used, a subset of a set may include one or more than one, including all, members of the set.

Further, if used in this disclosure, and unless stated or deducted otherwise, a first variable is an increasing function of a second variable if the first variable does not decrease and instead generally increases when the second variable increases. On the other hand, a first variable is a decreasing function of a second variable if the first variable does not increase and instead generally decreases when the second variable increases. In some embodiment, a first variable may be an increasing or a decreasing function of a second variable if, respectively, the first variable is directly or inversely proportional to the second variable.

The disclosed systems, methods, and apparatus are not limited to any specific aspect or feature or combinations thereof, nor do the disclosed systems, methods, and apparatus require that any one or more specific advantages be present or problems be solved. Further, any headings given in the description are not to be generally construed as being limiting on the subject matter disclosed under the heading, that is, the subject matter disclosed under one heading can be read in combination with the subject matter described under a different heading. In particular, unless specifically indicated otherwise, different aspects and embodiments of the inventions can be considered in combination if technically sensible. Any theories of operation are to facilitate explanation, but the disclosed systems, methods, and apparatus are not limited to such theories of operation.

Modifications and variations are possible in light of the above teachings or may be acquired from practicing the embodiments. For example, the described steps need not be performed in the same sequence discussed or with the same degree of separation. Likewise various steps may be omitted, repeated, combined, or performed in parallel, as necessary, to achieve the same or similar objectives. Similarly, the systems described need not necessarily include all parts described in the embodiments, and may also include other parts not described in the embodiments. Accordingly, the embodiments are not limited to the above-described details, but instead are defined by the appended claims in light of their full scope of equivalents. Further, the present disclosure is directed toward all novel and non-obvious features and aspects of the various disclosed embodiments, alone and in various combinations and sub-combinations with one another.

While the present disclosure has been particularly described in conjunction with specific embodiments, many alternatives, modifications, and variations will be apparent in light of the foregoing description. It is therefore contemplated that the appended claims will embrace any such alternatives, modifications, and variations as falling within the true spirit and scope of the present disclosure.

### Further Embodiments

The invention is further described by the following embodiments that are to be understood in the context of the entire present disclosure.

Embodiment 1: A system for transfer of energy inside a bodily lumen, the system comprising:
a stent-like implant configured to be positioned inside the bodily lumen; and
an energy emitting element configured to emit energy toward one or more target regions surrounding the bodily lumen, wherein:
the stent-like implant includes an energy conducting elements configured to have a predefined energy transfer function for allowing a controlled energy dose of the emitted energy reach the one or more target regions.

Embodiment 2: The system of embodiment 1 further comprising a pairing device configured to:
attach to the stent-like implant; and
deliver the stent-like implant inside the bodily lumen.

Embodiment 3: The system of embodiment 2, wherein the pairing device is configured to detach from the stent-like implant.

Embodiment 4: The device of embodiment 1, wherein the energy conducting element is configured to distribute the emitted energy so as to enhance energy intensity in one or more external locations substantially shielded by the stent-like implant from receiving the emitted energy.

Embodiment 5: The device of embodiment 1, wherein the energy conducting element is configured to distribute the emitted energy so as to decrease energy intensity in one or more external locations substantially overexposed to the emitted energy.

Embodiment 6: The system of embodiment 1, wherein the energy comprises light.

Embodiment 7: The system of embodiment 1, wherein the energy conducting element is configured to increase a uniformity of an energy distribution profile of the emitted light at the one or more target regions.

Embodiment 8: The system of embodiment 1, wherein the energy emitting element is configured to emit energy suitable for interaction with a substance administered to the one or more target regions.

Embodiment 9: The system of embodiment 8, wherein the substance is a member of at least one of the following groups:
a) a photosensitizer activated by energy from photons;
b) a photocatalyst activated by energy from photons;
c) a radiosensitizer activated by energy from radiation;
d) magnetic particles activated by magnetic energy; and
e) particles activated by energy from photons.

Embodiment 10: The device of embodiment 1, wherein the one or more target regions are configured to be located outside the bodily lumen.

Embodiment 11: The device of embodiment 10, wherein:
the one or more target regions contain diseased target tissues; and
the emitted energy is configured to damage at least a subset of the diseased target tissues.

Embodiment 12: The device of embodiment 11, wherein the diseased target tissues are located in proximity of the bodily lumen.

Embodiment 13: The device of embodiment 12, wherein the diseased target tissues are located within a distance in a range of zero to about 2-6 mm from a wall of the lumen.

Embodiment 14: The device of embodiment 11, wherein the diseased target tissues comprise cancerous tissues.

Embodiment 15: The device of embodiment 11, wherein the stent-like implant is configured to provide structural support for the lumen.

Embodiment 16: The device of embodiment 15, wherein the stent-like implant is configured to maintain structural integrity of the lumen following application of the energy to the diseased tissues.

Embodiment 17: The device of embodiment 1, wherein:
the stent-like implant is configured to be positioned inside a lumen of the body; and
the stent like implant is configured to provide structural support for the lumen.

Embodiment 18: The device of embodiment 1 wherein:
the stent-like implant includes a scaffolding structure; and
the energy conducting element is configured to reduce a shadowing effect caused by the scaffolding structure.

Embodiment 19: The system of embodiment 1, wherein the energy conducting element is configured to transmit through at least a portion of the energy configured to be emitted by the energy emitting element toward the one or more target regions.

Embodiment 20: The system of embodiment 19, wherein the energy conducting element is configured to reduce spatial inhomogeneities in intensity of the emitted energy external to the stent-like implant, or to tailor the system's energy emission function to the target region.

Embodiment 21: The system of embodiment 1, wherein the energy conducting element is configured to tailor the energy transfer function to the target region.

Embodiment 10: A method for transferring energy to target tissues inside a body, the method comprising:
positioning a stent-like implant inside a bodily lumen;
positioning an energy emitting element inside the bodily lumen;
emitting energy by the energy emitting element toward the target tissues, wherein:
the target tissues are located at one or more target regions surrounding the lumen, and
the emitted energy has a predefined energy emission function allowing for a controlled dose to be delivered to the target tissues.

Embodiment 23: The method of embodiment 22, further comprising:
administering a substance to the target tissues, wherein the substance is configured to interact with the emitted energy; and
activating the substance inside the target tissues by exposing the target tissues to the emitted energy, wherein activation of the substance causes damage to at least a subset of the target tissues.

Embodiment 24: The method of embodiment 23, wherein the substance is a member of at least one of the following groups:
a) a photosensitizer activated by energy from photons;
b) a photocatalyst activated by energy from photons;
c) a radiosensitizer activated by energy from radiation;
d) magnetic particles activated by magnetic energy; and
e) particles activated by energy from photons.

Embodiment 25: The method of embodiment 23, wherein activation of the PDT photosensitizer substance causes damage to at least a portion of the target tissues surrounding a vessel to facilitate resection of the target tissues while maintaining structural integrity of the vessel.
Embodiment 26: A method of treating diseased tissue inside a body, the method comprising:
administering an energy activatable substance to the body such that at least a portion of the energy activatable substance is introduced into the diseased tissue;
implanting a stent-like device in a portion of a lumen of a vessel in proximity of the diseased tissue; and
introducing an energy source into the lumen and positioning the energy source relative to the stent-like device such that at least a portion of the energy emitted by the source is transmitted through a portion of the stent-like device for irradiating at least a portion of the diseased tissue so as to activate the substance, thereby damaging at least a portion of the diseased tissue,
wherein the stent-like device is configured to maintain structural integrity of the vessel during treatment.

Embodiment 27: The use of a substance/composition of matter that can be activated with energy (e.g. a PDT photosensitizer, radiosensitizer, magnetic particle etc.) for damaging at least a portion of a diseased tissue surrounding a lumen or vessel to facilitate surgical resection with disease-free margins around the lumen or vessel while the energy is delivered via the endoluminal/endovascular route and the structural integrity of the lumen of vessel is maintained.

### EXAMPLES

The following working examples of the invention further illustrate the invention and are in no way to be construed as limiting on the invention.

Various cancers can surround and invade blood vessels, such as large blood vessels, resulting in complicated or even prevent, radical surgical resection with demonstrable tumor free margins. Hence, these technical challenging procedures present a clinical management problem, especially for pancreatic cancers. In the USA alone, 55,000 patients were newly diagnosed with pancreatic cancer in 2018. Of these, about 30% present with locally advanced disease and are deemed non-eligible for surgery because of vessel involvement. Clearing the blood vessels from tumor involvement, and thus clinically downstaged patients to become eligible for surgery would be highly beneficial as it may ultimately improve chances for prolonged survival.

Several downstaging approaches are under investigation, including combinations of systemic chemotherapy and radiation therapy. Presently investigated downstaging approaches in the art come at the cost of local and systemic toxicities. Additionally, not all patients are effectively downstaged, and the typical treatment duration can last weeks or even months, with reduced quality of life during this interval.

Thus, a more effective local treatment modality with short treatment duration remains desirable. Proposed methods include radiofrequency and microwave application, cryotherapy, irreversible electroporation, electrochemotherapy, brachytherapy, high-intensity focused ultrasound, and laser thermal therapy. However, it remains challenging to ablate tissue, such as diseased tissues, e.g. cancer tissue, effectively around a vessel due to the inherent anatomical complexity, and the need to preserve the vessel function.

To overcome these challenges, we herein propose to deliver the therapeutic energy delivery via the endovascular route. However, it is not well understood if a sufficient therapeutic energy dose, can be delivered via the vessel wall into the surrounding tumor tissue to achieve the surgical required tissue clearing without comprising the vessel function or causing severe complications including vessel perforation, stricture, or thrombus formation.

### Example 1: Monte Carlo Light Dose Simulations

Monte Carlo simulations were performed to assess if a sufficient therapeutic mediated photodynamic therapy (PDT) dose of Verteporfin (also known as benzoporphyrin derivative monoacid ring A or BPD-MA) can be delivered to the target tissues surrounding the vessel.

In these simulations, we assumed intravenous administration of a light-absorbing drug and the placement of a light emitter in the endovascular system at the site of tumor encroachment around it. Light absorbed by the photosensitizer was assumed to result in the local creation of short-lived cytotoxic radicals, causing cell death e.g. hemorrhagic tissue necrosis and apoptosis. To quantify the attainable effects, light-dose escalation studies were performed using Monte Carlo light propagation simulations (FullMonteCUDA) (“High-performance, robustly verified Monte Carlo simulation with FullMonte,” J. Biomed. Opt. 23(8), 085001 (2018)). The use of the photosensitizer BPD-MA mediated PDT was initially investigated in a simple “cube and tube” geometry representing the pancreas and a vessel traversing its center. The multilayered tube represented the vascular wall layers, intima, media and adventitia having an air-filled balloon with a linear line source in the center. The superior mesenteric and the splenic arteries were selected as model systems because of their frequent involvement in locally advanced pancreatic cancer. Simulation properties were assigned as described above and PDT activation energies were escalated over the range of 10-100 J cm⁻¹ diffuser length, and the anticipated threshold dose in mJ·cm⁻³ and radii of necrosis were estimated based on the photodynamic threshold model introduced by Patterson M, Wilson B C, Graff R: In vivo test of the concept of photodynamic threshold dose in normal rat liver photosensitized by aluminum chlorosulfunated phthalocyanine. Photochem Photobiol, 51(3): 343-349, 1990.

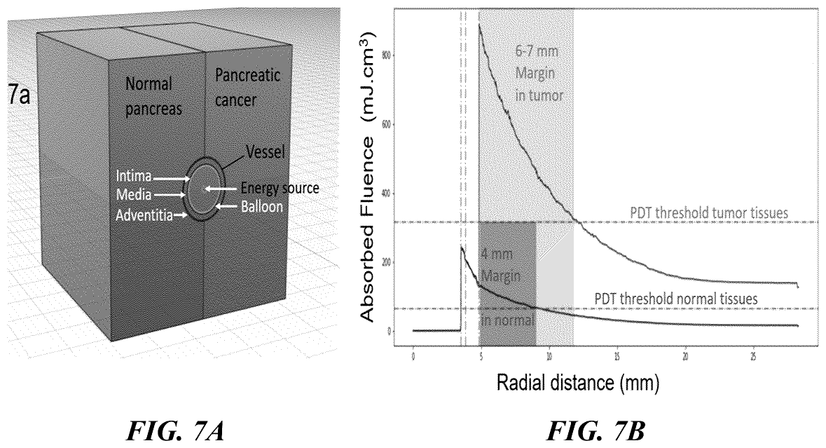

The pancreatic model is shown in FIG. 7A comprising a 4×4×5 cm cube, one half is assigned with optical properties of normal pancreatic tissue and the other half with those of cancerous pancreatic tissues. Running through the cube's center is a blood vessel made up of three layers, intima, media, adventitia, with assigned properties from the literature for aortic tissues. An initial dose-escalation simulation investigated if an adequate fluence [J·cm⁻²] can be delivered through the vessel wall to create a surgically sufficiently large circumferential necrotic margin (mm) surrounding the vessel, based on the photosensitizer accumulation and the sensitizer's molar extinction coefficient. The vessel's inner diameter and wall thickness were set to 7.0 mm and 1.30 mm, respectively. The energy emitting source was cylindrical in shape with a length of 20 mm and the irradiance at the intima surface was fixed at 200 mW·cm⁻². The exposure times escalated to deliver 10 to 100 J total energy. An example of the absorbed energy density as a function of radius from the vessel center is shown in FIG. 7B, with an estimated necrotic margin ranging from 4-7 m. The subsequent necrotic clearing of viable tumor tissue from the adventitia is predicted based on the threshold model for common photosensitizers, see Tables 1 and 2.

TABLE 1
Estimated PDT threshold doses for several photosensitizers

| | | Molar | | | | |
|---|---|---|---|---|---|---|
| | | extinction | | Photo- | | Critical |
| | Molar | coefficient | Tumor | sensitizer | | absorbed |
| Photosensitizer@ | weight | ϵ | uptake | concentration | 2.3 ϵC | energy |
| Wave-Length [nm] | [g · mol⁻¹] | [M⁻¹ cm⁻¹] | [mg · g⁻¹] | [M] | [cm⁻¹] | [mJ · cm⁻³] |
| Phtalocyanine@690 nm | 514.5 | 7,0704 | 12.3 | 2.3 · 10⁻⁵ | 3.25 | 2,345 |
| Verteporfin@689 nm | 718.8 | 33,000 | 23.9 | 3.3 · 10⁻⁵ | 2.50 | 3 |
| Talaporfin@664 nm | 711.8 | 40,000 | 1.62 | 2.2 · 10⁻⁶ | 0.20 | 1,500 |
| Temoporfin@652 nm | 680.7 | 30,000 | 0.5 | 7.9 · 10⁻⁷ | 0.05 | 73 |
| Photofrin@630 nm | 1179.4 | 1,170 | 8.1 | 6.8 · 10⁻⁶ | 0.02 | 6,864 |
| ALA-PpIX@630 nm | 562.7 | 5,121 | 10.3 | 1.8 · 10⁻⁵ | 0.22 | 376 |

TABLE 2
The estimated circumferential margin of necrosis (mm) as measured from
the center of a 7 mm diameter vessel, for several photosensitizers as function of light energy.

| Photo- | 10 J | | 20 J | | 40 J | | 80 J | | 100 J | |
|---|---|---|---|---|---|---|---|---|---|---|
| sensitizer | Normal | Tumor | Normal | Tumor | Normal | Tumor | Normal | Tumor | Normal | Tumor |
| Phthalo- | 0 | 1.2 | 1.2 | 2.2 | 2.0 | 3.2 | 3 | 4.2 | 4.1 | 5.0 |
| cyanine | | | | | | | | | | |
| Verteporfin | 9.5 | 9.5 | 12.7 | 12.7 | 20.2 | 20.2 | 25.0 | 25.0 | >25.0 | >25.0 |
| Talaporfin | 0 | 0 | 0 | 0 | 0 | 1.0 | 0.1 | 5.0 | 2.0 | 7.5 |
| Temoporfin | 0 | 2 | 1.5 | 7.4 | 7.2 | 15.0 | 10.2 | 19 | 14.2 | >25.0 |
| Photofrin | 0 | 0 | 0 | 0 | 0 | 0 | 0 | 0 | 0 | 0 |
| ALA- | 0 | 0 | 0.2 | 3.4 | 3.2 | 7.5 | 7.2 | 12.5 | 11.2 | 14.4 |
| PpIX | | | | | | | | | | |

From Table 2 it becomes apparent that a circumferential margin of necrosis in pancreatic cancerous tissues around the modelled vessel is feasible with several photosensitizers. The dimension of this margin is estimated between circa up to and beyond 25 mm for doses ranging from 10-100 J.

For Verteporfin (BPD-MA), based on the light dose escalation carried out here, circumferential tissue necrosis could be anticipated (e.g. around the superior mesenteric artery) with a margin diameter ranging from about 10 mm to 25 mm (measured from the center of the vessel) with the assumed tissue uptake in normal and malignant tissues for BPD-MA mediated PDT. At clinically acceptable irradiation times in the range of several minutes and an intimal irradiance of up to 200 mW·cm⁻² thermal damage to the vessel wall is considered unlikely for red and NIR wavelengths.

### Example 2: In-Vivo Porcine Model

### Hybrid Operating Room, Equipment, and Tools

For anatomical and vascular assessment, non-enhanced and contrast-enhanced CT scans were performed just before the illumination procedures. To guide the interventional procedures, a robotic angiography system and ultrasound system were used (SOMATOM Definition AS+, Artis zeego, ACUSON 53000, Siemens Healthineers AG, Germany).

### Porcine Model

Five large white porcine (Sus scrofa domesticus), weighing between 25-43 kg, were used as animal model, because the anatomy of the porcine pancreas and its vasculature closely resembles that of the human. Animals were handled according to the European Directive 2010/63 and French laws concerning animal protection in laboratories.

The animals were housed in a group and acclimatized for 48 hours in an enriched environment, respecting circadian cycles of light-darkness, and with constant humidity and temperature conditions. They fasted 24 h before the intervention, with ad libitum access to water, and finally sedated (zolazepam+tiletamine 5-10 mg/kg IM) (Zoletil®, Virbac) 30 min before the procedure and transfer to the operative room.

Following the intravascular procedure, porcine recovered from anesthesia and were housed for a survival period of 24 hours up to one week. After the survival period the sedation protocol above was repeated and general anesthesia was induced using intravenous (18 G Intravenous catheter in-ear vein) propofol 3 mg/kg, followed by orotracheal intubation and maintained with rocuronium 0.8 mg/kg along with inhaled isoflurane 2%. At the end of the experiment, animals were euthanized with an intravenous injection of pentobarbital 40 mg/kg IV (Exagon ND, Axience).

### Premedication

The preclinical experiments in pigs required some precautions. To counteract possible effects of BPD-MA on the porcine blood pressure, the antihistamine dexchlorpheniramine was given the days before and immediately prior to the start of photosensitizer infusion. Polaramine® was administered at 2 mg p.o. BID to TID the day before and Polaramine® 5 mg IV (slow bolus) 30 to 60 minutes before photosensitizer injection. Methylprednisolone SoluMedrol®120 (3 mg kg-1) was administered also IV (slow bolus) 30 to 60 minutes before photosensitizer injection. In addition, a 5% dextrose IV drip was used rather than saline during the injection period and for 60 min thereafter to prevent possible BPD-MA aggregation. Heparin was administered in a single dose to avoid hypercoagulability complications (50 UI/kg in bolus IV).

The porcine was prepared for intervention by an intramuscular injection of zolazepam+tiletamine 5-10 mg·kg⁻¹ IM (Zoletil®, Virbac) as a sedative prior to the transfer to the operative room. One peripheral vascular access (auricular vein) was placed for anesthesia. A second one was placed on the other ear and used for the BPB-MA administration. The central access (jugular vein) was placed for emergency injections, including adrenaline by pump 2 mL (kg·hr−1), or rapid vascular filling in the case of a cardiovascular failure. 30 minutes after sedation, anesthesia induction was performed with IV propofol 2.5-3 mg kg-1 (Propovet®, Zoetis) and rocuronium 1-1.5 mg kg-1 (Esmeron®, MSD). This allowed the pig to be intubated with a tracheal tube (6.0). To maintain anesthesia, isoflurane 2-3% (Iso-Vet®, Piramal Critical Care) was delivered in a mix of oxygen and air while the animal was maintained under mechanical ventilation.

A saline drip was maintained throughout the duration of the anesthesia. Analgesia was ensured by injection of buprenorphine 0.01 mg kg-1 IV (Buprecare® 0.3 mg/ml, Axience). Non-steroidal anti-inflammatory drugs were not used, as they may interfere with an intended inflammatory effect due to the treatment. The animal received Amoxicillin/Colistin 10 mg-25000UI kg-1 IM (Potencil® Virbac, 1 ml (10 kg)-1 as antibiotics.

Pre-Treatment Planning CT and Co-Localization with Angiography

To visualize the pancreas and its target vessels for treatment planning, non-enhanced and contrast-enhanced CT scans were carried out shortly before treatment, in the early arterial and early portal venous phase. The position, morphology, and dimensions of the pancreas, the superior mesenteric artery (SMA), splenic and hepatic artery were assessed as well as the veins. Suitable target areas were selected where the vessel was surrounded by pancreatic tissues. A 3D vascular roadmap was obtained via software manipulation (syngo.via, Siemens Healthineers AG, Germany) and the position, morphology and dimensions of the target vessels were assessed and used for later co-localization during the angiography-guided procedure and placement of a suitably sized PDT light source, being a prototype irradiating balloon catheter.

### Photosensitizer Infusion

Following the planning CT, animals were moved to a hybrid OR for photosensitizer infusion. BPD-MA is a regulatory approved product for the treatment of age-related macular degeneration (AMD). It is under investigation by several groups for the treatment of a variety of cancers, including pancreatic cancers. It was administered here at doses of 0.4 mg kg⁻¹ or 1.6 mg kg⁻¹ to animals via a slow intravenous infusion under supervision by a vet. The infusion rates increased progressively up to 1 ml·min⁻¹ over a period of circa 10-15 min using a syringe pump (Harvard Apparatus, Model 22, USA). The infusate was filtered by a 0.2 μm syringe filter. Dedicated light opaque syringes and injection tubing were used to protect the medication from light exposure. Close monitoring of gas anesthesia, body temperature and vital parameters was ensured via an anesthesia machine (Primus Infinity®, Drager and the Maglife Serenity RT1®, Schiller, France). The animal was kept under dimmed ambient lighting to prevent possible skin photosensitization.

### Endovascular Procedure

A 6-7 French prototype irradiating balloon catheter was used for endovascular energy delivery to the target vessels and guided into position using fluoroscopy. To place the irradiating balloon into the target vessels, either the femoral access route or the percutaneous transhepatic route was used for access to the target vessels. In both cases, the Seldinger technique was used to place an 8 French angiography introducer sheath (Terumo Europe NV, Belgium) under aseptic conditions. A 5 French Cobra 2 catheter C (Terumo) was inserted, followed by a 0.035″ hydrophilic guidewire over which the irradiating balloon catheter was placed in the target vessels being the splenic artery, hepatic artery, and superior mesenteric artery.

### Light Source and Light Delivery

The required energy for PDT was generated by a 690 nm diode laser with up to 2 W output. The power output was calibrated using the internal integrating sphere that was cross calibrated against NIST-traceable power meters. The laser power was coupled into an optical fiber having a 400 μm core diameter and a 1 cm long cylindrical diffuser tip, which was centrally placed inside of the prototype irradiating balloon catheter (Virtual Biotech, Germany) for homogenous irradiation of the vessel inner wall and radially outwards into the surrounding tissues. The balloon itself had a length of 3 cm and variable diameters ranging between 4-8 mm to accommodate different vessel diameters (materials provided by Virtual Biotech, Germany). The laser power was adjusted to achieve an intimal wall surface irradiance of 150 mW·cm⁻². Case 1 was injected with 0.2 mg·kg⁻¹ BPD-MA and was not irradiated. Cases 2-3 were injected with 0.4 mg·kg⁻¹ BPD-MA, the irradiation time was up to 25 min, delivering up to 225 J·cm⁻². Cases 4-5 were injected with 0.8 and 1.6 mg·kg⁻¹ BPD-MA had an irradiation time of 8.3 min to deliver 75 J·cm⁻². Light irradiation started about 60 minutes after the end of the BPD administration and ended circa 90 minutes later. After treatment, animals were allowed to recover and remained protected from direct bright light exposure for 48 hrs. During the recovery period, the animals were monitored by a veterinarian or treating physician 3 times per day and hourly in the early postoperative periods.

### Assessment of Treatment Response

At 24 h up to one week post endovascular PDT the animals underwent in-vivo contrast-enhanced CT scanning as described above and density changes within the pancreas were assessed and compared with the baseline CT scans. After this CT scan, the animals were sacrificed. At necropsy, the pancreas was surgically removed en bloc. At necropsy, target vessels treated with PDT together with the surrounding pancreatic tissues were dissected into circa 1 cm³ blocks, photographed and fixed in 10% neutral buffered formalin. A suture was fed through the vessel lumen to maintain anatomical orientation. Sections were stained with standard H&E staining to examine PDT induced histopathological changes to the pancreas and its adjacent tissues, and, to examine the structural integrity of the vessel wall.

### Results

The first porcine received an i.v. saline drip and was injected with BPD-MA, without above-described pre-medications. Immediate changes occurred in the heart rate, decreasing to a degree that adrenaline was administered to stabilize the animal and the photosensitizer injection was stopped early at 0.2 mg kg⁻¹. The animal gradually recovered after 10 to 20 min and stabilized to normal heart rates. Since the animal did not receive the desired drug dose of 0.4 mg kg⁻¹ as per protocol—the animal was sacrificed at one hour after photosensitizer injection.

Porcine 2-3 were pre-medicated as described above, excluding, heparin, received a 5% i.v. dextrose drip and was injected with 0.4 mg·kg⁻¹ BPD-MA. No changes occurred in blood pressure or heart rate during the injection. Target vessels were selected for treatment, being the splenic artery and vein, the hepatic artery, and the superior mesenteric artery. Vessels were treated with an irradiance of 150 mW·cm⁻² at the inner vessel lumen. This resulted in a dose of up to 225 J·cm⁻² over a 25 min exposure time, utilizing a first-generation prototype irradiating catheter with a compliant balloon, having a variable diameter with variable balloon inflation pressure.

Porcine 4-5 were pre-medicated as described above including heparin, received a 5% i.v. dextrose drip and was injected with 1.6 mg·kg⁻¹ (N=2) BPD-MA. No changes occurred in blood pressure or heart rate during the injection. Target vessels were the same as above and were treated with a dose of 75 J·cm⁻² over an 8.3 min exposure time, utilizing a second-generation prototype irradiation catheter with a non-complaint balloon, having a rather fixed diameter with variable balloon inflation pressure.

Case 2 underwent a CT scan at 24 h, case 3 at 48 h and cases 4-5 at day 3 and one week post treatment prior to sacrifice.

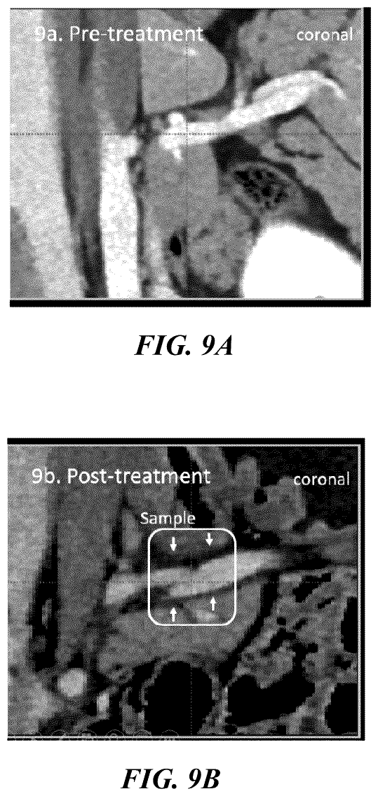

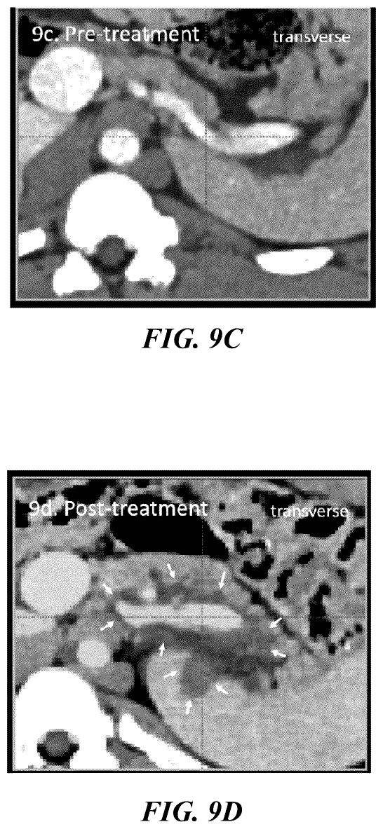

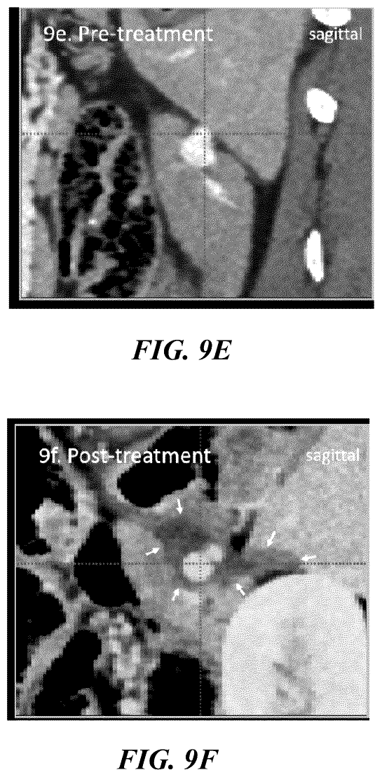

As assessed by a radiologist, on the contrast-enhanced CT scans, the vasculature was able to withstand these relatively high doses while substantially maintaining the vessels' structural integrity, at least to a degree that no perforations, ruptures, and bleedings were observed during this study. The target vessels demonstrated subtle areas of density changes in the areas surrounding the target vessels, as compared to the baseline CT scan. The CT images in perivascular tissues directly around the treated vessel appeared more diffusely vs. baseline and was interpreted as possible fluid accumulation and swelling possibly due to inflammation. The pancreatic parenchyma adjacent to the target vessel appeared more hypodense vs. baseline. This hypodense area appeared circumferentially around the target vessel radially extending outwards with a dimension of approximately 2-6 mm extending outwards from the outer wall of the vessel and partially into the spleen. The observed hypodensity may be interpreted as possible PDT-induced necrosis within the pancreas. These hypodense areas are indicated by white arrows in FIGS. 9A-F that is showing the splenic artery in sagittal, coronal, and transverse views on pre- and post-treatment CT scans in the early portal venous phase.

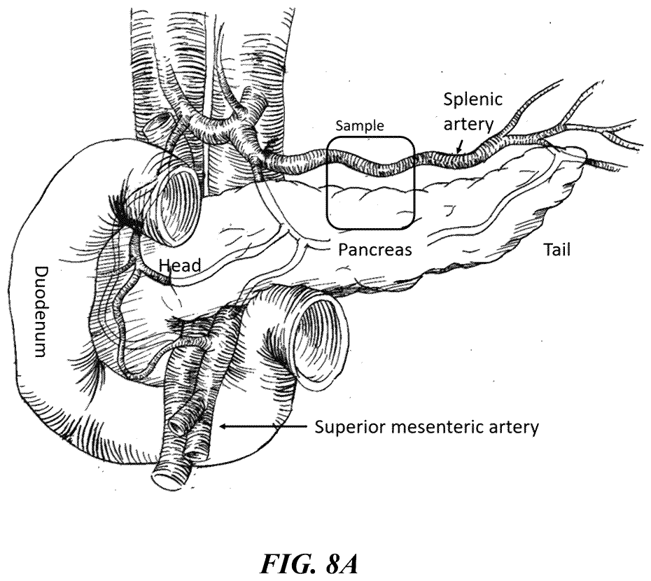

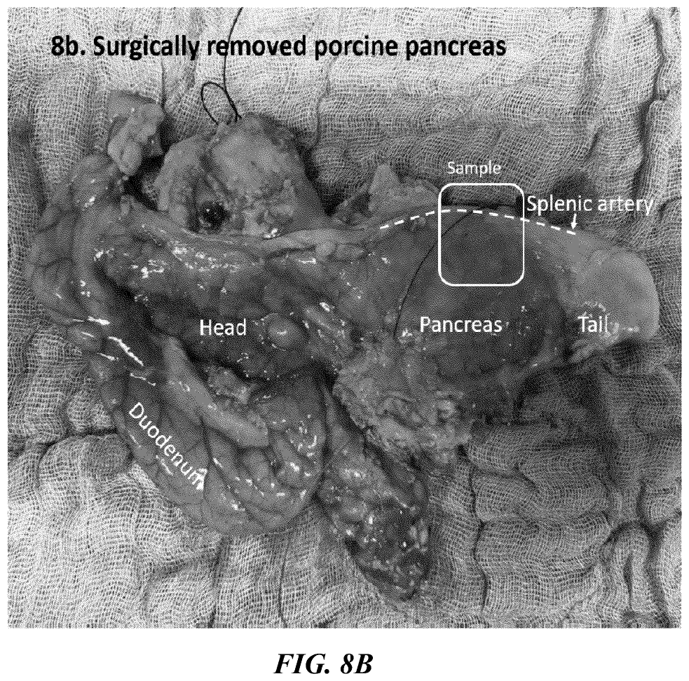

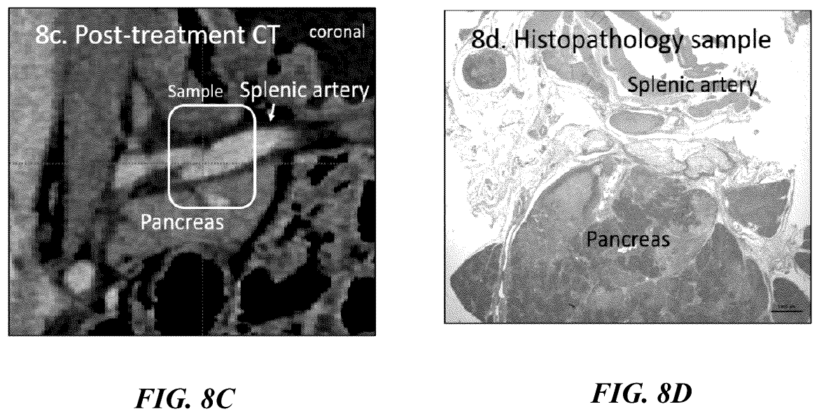

Upon removal of the pancreas, the operating surgeon felt palpable softer spots in the pancreas in the areas subjected to treatment. Upon dissecting the target areas with a scalpel in 1 cm³ blocks there were no visible signs of vasculature rupture, yet within the treatment area some of the vessel wall appeared more pale and white-ish in color extending radially outwards into the pancreas, assumed to be the PDT treatment effect up to 7 days after treatment. A sketch is provided for anatomical orientation (FIG. 8A) and a picture of the excised pancreas can be seen in FIG. 8B indicating the area of treatment in the splenic artery, where the sample was taken.

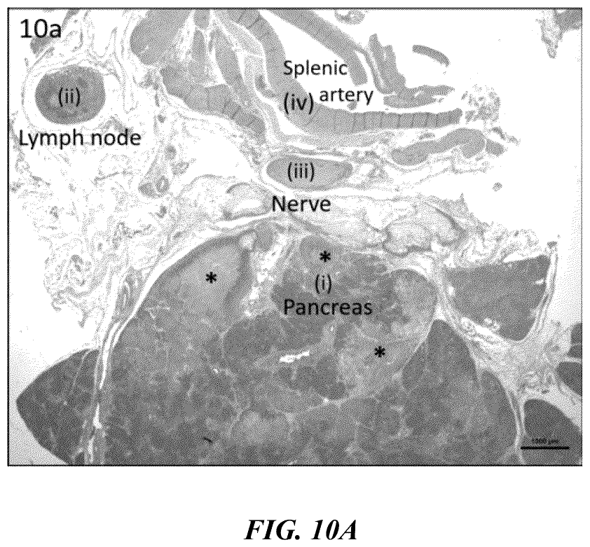

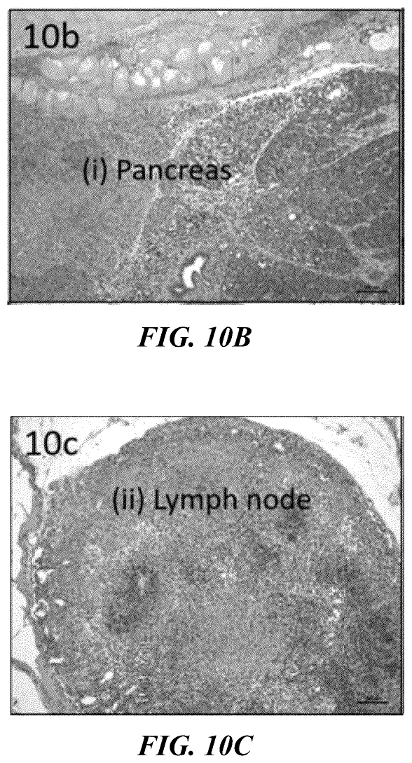

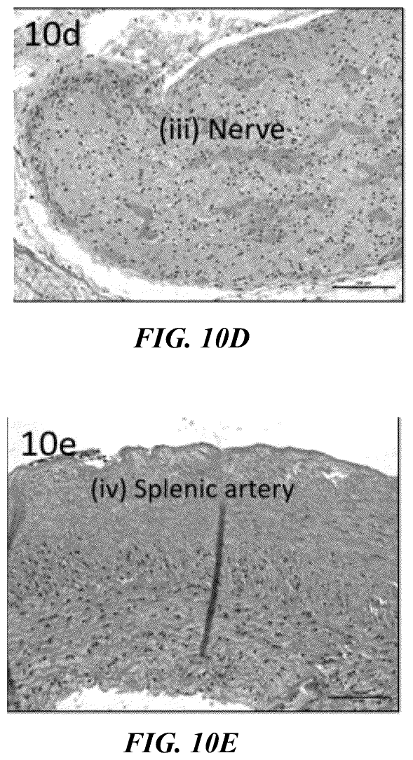

Histopathology processing was carried out on the selected specimens. Embedding, cutting (3 μm) and hematoxylin & eosin (HE) staining was performed and the slides assessed by a histopathologist. To provide a general overview, FIG. 10A shows a low magnification image as to demonstrate the entire topography of the different tissue structures within the sample. The scale bar is 1 mm. One can see the splenic artery (iv) running horizontally atop the pancreas (i). To the left is a lymph node (ii) and in between the splenic artery and pancreas, nerve (iii) tissue can be found. For easy comparison, this sample is co-localized and provided in the same view and orientation as the sketch and picture of the surgically removed pancreas in FIG. 8A-B as well as the pre- and post-treatment CT images in FIG. 9A-F.

In the pancreas adjacent to the target vessel, multifocal necrosis, apoptosis and degenerative changes were recorded and indicated with several * asterisk signs in FIGS. 10A-C. Similar results were found in the surrounding lymph node (ii) and nerves structures (ii). This is viewed in more detail at higher magnification in FIG. 10B, showing both liquefactive pancreatic necrosis as well as fat necrosis, in FIG. 10C showing liquefactive necrosis of the adjacent lymph node and in FIG. 10D demonstrating hemorrhagic necrosis within the nerve tissues. The observed necrosis and tissue degeneration extended about 6 mm radially outward from the treated vessel, match with CT findings, were not observed in untreated control tissues, and could be linked to PDT treatment.

In the perivascular tissues directly around the treated vessel mononuclear inflammatory infiltrates were observed, which correspond with the perivascular changes observed with CT. When examining the splenic arterial wall (iv) in FIG. 10E, endothelial loss and necrotic cellular tissues were observed, but the extracellular matrix containing collagen and elastin was preserved in some cases, essentially creating an acellular or decellularized vascular scaffold (FIG. 10E).

In contrast-enhanced CT, the pancreatic parenchyma adjacent to the target vessel appeared hypodense vs. baseline, appeared circumferentially around the target vessel, radially extending outwards circa 2-6 mm. The observed hypodensity can be interpreted as PDT-induced necrosis within the pancreas. The vasculature was able to withstand these relatively high doses while substantially maintaining the vessels' structural integrity, at least to a degree that no perforations, ruptures, and bleedings were observed in this study and cases 5 and 6 survived for 7 days after the treatment demonstrating normal behavior.

Histopathological examination demonstrated liquefactive necrosis in the pancreas, nerve tissues and lymph nodes tissues adjacent to the target vessel and circumferentially around it radially extending outwards up to approximately 6 mm away from the target vessel, in-line with CT observations.

### Example 3: PDT Dose Simulation

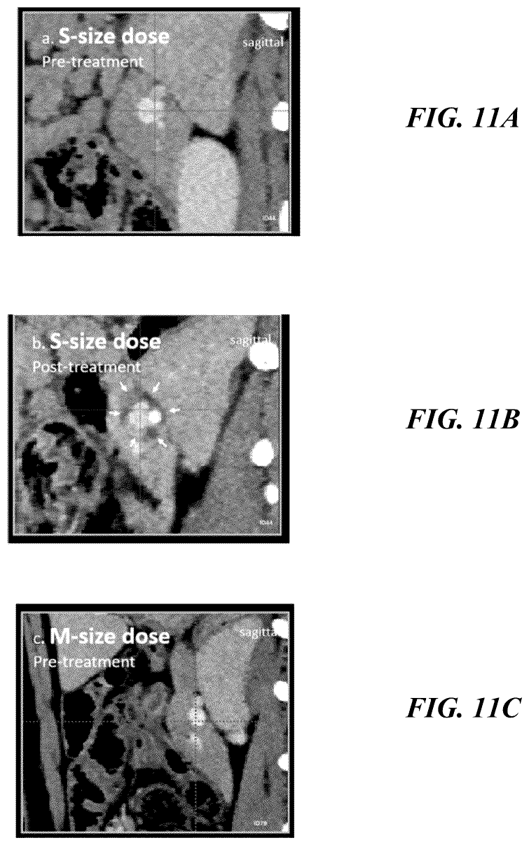

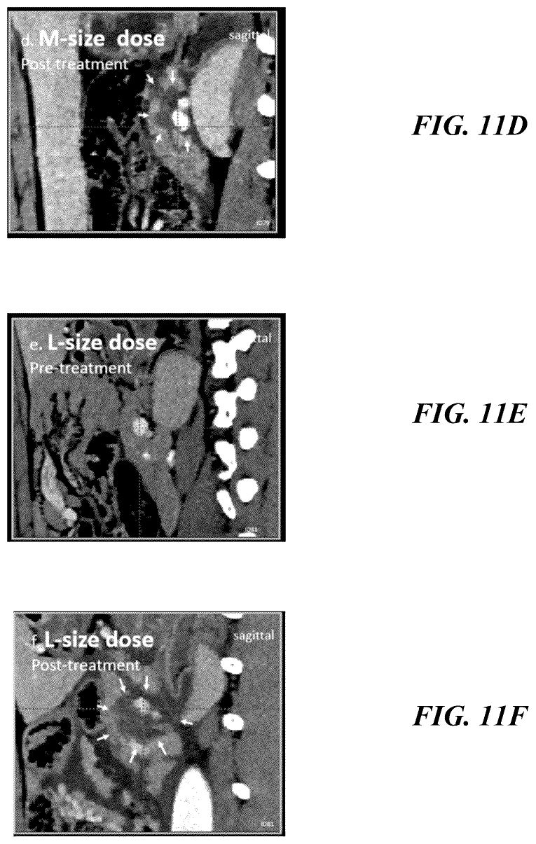

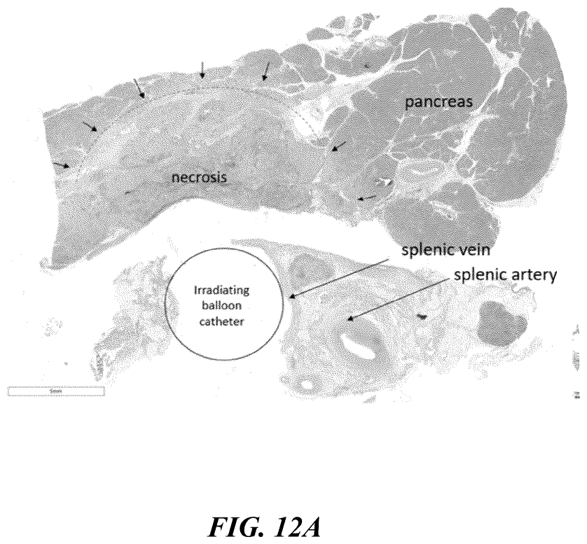

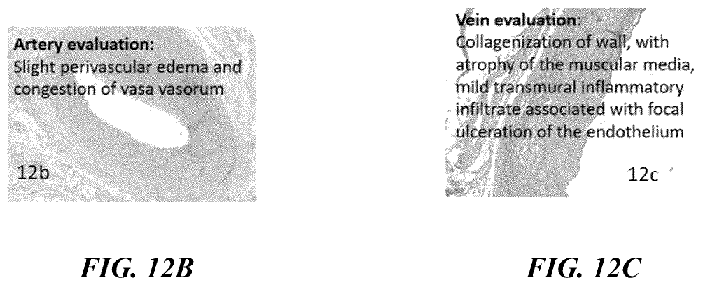

The performance of Monte Carlo simulations determined that the photosensitizer verteporfin appears to be a suitable photosensitizer and that perivascular necrosis can occur when irradiating at relevant light dose ranges in a clinically acceptable time range of several minutes. In-vivo experiments were carried out in healthy porcine (n=7) delivering light irradiation from an endovascular balloon-catheter laser probe. These survived for 2, 3, or 7 days and demonstrated that following a dose escalating administration of verteporfin (0.4-3.2 mg/kg) and light irradiation (8-25 minutes), resulted in increasing radius of apparent perivascular circumferential tissue necrosis of circa 2-15 mm, as assessed by 48h contrast-enhanced CT (FIGS. 11A-F) and histopathology (FIGS. 12A-C).

FIGS. 11A-F shows pre-treatment (FIGS. 11A, 11C, 11E) and post-treatment at 48 h (FIGS. 11B, 11D, 11F) contrast enhanced CT demonstrating a sagittal view of a porcine pancreas for escalating doses of S (0.4 mg/kg; FIGS. 11A and 11B), M (0.8 mg/kg; FIGS. 11C and 11D), and L size (1.6 mg/kg; FIGS. 11E and 11F). The irradiating balloon catheter was placed in the splenic vein (crosshair) and an increasingly large hypodense margin, interpreted as PDT induced necrosis, can be observed for increasing doses.

FIGS. 12A-C shows the histopathological evaluation 48h post PDT treatment (H&E and EVG stained), representative for an animal treated with an L-size dose (1.6 mg/kg). In FIG. 12A, the pancreas can be observed with the splenic vein, artery, and the approximate location of the irradiating balloon catheter. Radially extending into the pancreas is a circumferential margin of necrosis (dotted line and arrows, circa 10 mm). FIGS. 12B-C are magnifications of the artery and vein evaluations demonstrating intact vessel unruptured walls albeit with atrophic muscular cells and ulcerated endothelial cells. The artery evaluation of FIG. 12B shows slight perivascular edema and congestion of the vasa vasorum. The vein evaluation of FIG. 12C shows collagenization of the wall, with atrophy of the muscular media and mild transmural inflammatory infiltrate associated with focal ulceration of the endothelium.

Based on in-silico simulation, PDT-dose simulation, and in-vivo porcine model experiments as described above, it is demonstrated that relevant circumferential margins of necrosis can be produced around a vessel surrounded by pancreatic tissue, utilizing PDT via the endovascular route, while not causing perforations, ruptures, and bleeds up to 7 days after treatment. This was demonstrated by histopathological analysis and visualized with CT. These attainable margins are expected to be sufficient for a surgeon to operate on pancreatic cancer patients, previously deemed inoperable, thereby downstaging the patient.

Those having ordinary skill in the art will appreciate that various changes can be made to the above embodiments without departing from the scope of the invention.

1.-124. (canceled)

125. A method for isolating a biological vessel from a surrounding target tissue comprising:
administering an energy-activatable substance to at least a portion of the target tissue; and
activating the energy-activatable substance via intraluminal irradiation of the portion of the target tissue with the energy-activatable substance.

126. The method of claim 125, further comprising:
introducing an energy-emitting element into the vessel, wherein the energy-emitting element is configured to emit radiation that irradiates the at least a portion of the target tissue with the energy-activatable substance.

127. The method of claim 126, wherein the energy-emitting element emits energy that is below a vessel wall damage threshold and above a damage threshold for surrounding target tissue.

128. The method of claim 127, wherein the damage threshold to surrounding target tissue is a photochemical threshold.

129. The method of claim 127, wherein the vessel wall damage threshold is any of a thermal and a photochemical threshold.

130. The method of claim 126, wherein the energy-emitting element is further configured to emit the radiation at a wavelength and a fluence such that the emitted radiation can activate the energy-activatable substance while maintaining a structural integrity of the vessel wall substantially intact.

131. The method of claim 126, further comprising:
protecting the vessel from structural damage before and/or during and/or after the energy-emitting element emits radiation for activating the energy-activatable substance.

132. The method of claim 131, wherein the step of protecting the vessel from damage includes temporarily compressing vasa vasorum before and during the energy-emitting element emits radiation so as to cause temporary deprivation of the irradiated vessel wall from oxygen, wherein optionally the step of protecting the vessel from structural damage includes inducing collagen cross-linking in the irradiated vessel wall thereby creating an in-situ vascular scaffold.

133. The method of claim 132, wherein the step of protecting the vessel from structural damage includes placing an endoluminal wall protection device within the vessel, and wherein optionally the endoluminal wall protection device is any of a stent-like device and a balloon.

134. The method of claim 125, wherein the radiation has a predefined energy emission function.

135. The method of claim 134, wherein the predefined energy emission function allows for a controlled dose of energy to be delivered to the target tissue with the energy-activatable substance.

136. The method of claim 125, wherein the at least a portion of the target tissue is in contact with a portion of an external surface of the vessel and the activation of the energy-activatable substance generates cell death that effects isolation of a part of the at least a portion of the target tissue from the vessel surface.

137. The method of claim 136, wherein the cell death allows subsequent surgical isolation of a tumor from the vessel.

138. The method of claim 125, wherein the energy has an intensity distribution profile so as to irradiate the at least a portion of the target tissue so as to cause the isolation in an at least partly circumferential pattern around the vessel surface.

139. The method of claim 125, wherein the structural integrity of the vessel wall remains substantially intact subsequent to isolation from the portion of the target tissue.

140. The method of claim 125, wherein the target tissue includes diseased cells.

141. The method of claim 125, wherein the radiation has a wavelength between 500 and 1500 nm and wherein the radiation irradiates an inner wall of the vessel at an irradiance less than about 200-600 mW·cm⁻².

142. The method of claim 125, wherein radiation deposits a total energy between 1 and 1000 Joules·cm⁻² at an inner wall of the vessel.

143. The method of claim 125, wherein the energy-activatable substance comprises a photosensitizer, a photocatalyst, a radiosensitizer, a sonosensitizer, magnetic particles, or nanomaterials.

144. The method of claim 143, wherein the photosensitizer is selected from the group consisting of porphyrins, chlorins, bacteriochlorins, and phthalocyanines, or a pharmaceutically acceptable derivative thereof selected from pharmaceutically acceptable salts, esters and amides, and wherein optionally the photosensitizer is verteporfin, talaporfin, padeliporfin or temoporfin, or a pharmaceutically acceptable derivative thereof selected from pharmaceutically acceptable salts, esters and amides.

145. The method of claim 125, wherein the energy-activatable substance includes a targeting moiety, and wherein optionally the targeting moiety includes an antibody, an antibody fragment, a peptide, an aptamer, or a small molecule.

146. The method of claim 125, wherein the energy-activatable substance is administered orally, intra-luminally, intra-arterially, intravenously to the subject, or is injected directly into the target.

147. The method of claim 125, wherein the energy-emitting element is configured such that the emitted radiation and activated substance induces the collagen cross-linking in the irradiated vessel wall.

148. A method of treating a patient suffering from cancer, comprising:
administering an energy-activatable substance to at least a portion of the target tissue of an organ; wherein the at least a portion of the target tissue is in contact with a portion of an external surface of a vessel of the organ,
activating the energy-activatable substance via intraluminal irradiation of the portion of the target tissue containing the energy-activatable substance to effect isolation of at least a part of the at least a portion of the target tissue from the vessel surface, and
causing any of removal or necrosis of at least some of the isolated part of the at least a portion of the target tissue to treat cancerous tissue.

149. The method of claim 148, wherein structural integrity of the external surface of the vessel of the organ remains intact.
